(19) United States
(12) Patent Application Publication
(10) Pub. No.: US 2014/0303198 A1
(43) Pub. Date: Oct. 9, 2014

(54) AGENT FOR INHIBITING EXPRESSION OF NPC1L1 AND/OR LIPG MRNA AND AGENT FOR PREVENTING AND/OR TREATING OBESITY

(71) Applicant: KOWA COMPANY, LTD., Nagoya-shi, Aichi (JP)

(72) Inventors: Takeshi Murakami, Tokorozawa-shi (JP); Kentaro Murakami, Boston, MA (US); Tadaaki Ohgiya, Tokorozawa-shi (JP); Kimiyuki Shibuya, Tokorozawa-shi (JP)

(73) Assignee: KOWA COMPANY, LTD., Nagoya-shi, Aichi (JP)

(21) Appl. No.: 14/358,399

(22) PCT Filed: Nov. 28, 2012
(86) PCT No.: PCT/JP2012/080731

§ 371 Date: May 15, 2014

(30) Foreign Application Priority Data
Nov. 29, 2011 (JP) 2011-260334

(51) Int. Cl. A61K 31/505; A61K 31/421

(52) U.S. Cl. CPC A61K 31/505; A61K 31/421
USPC 514/272; 514/376

(57) ABSTRACT

Provided is an agent for treating and/or preventing obesity. An agent for suppressing expression of NPC1L1 (Niemann-Pick disease, type C1, gene-like 1) and/or LIPG (Lipase, endothelial) mRNA, and an agent for preventing and/or treating obesity, each comprising, as an active ingredient, a compound having CETP inhibitory activity, or a salt thereof, or a solvate thereof.

### TECHNICAL FIELD

The present invention relates to an agent for preventing and/or treating obesity. In addition, the present invention relates to an agent for suppressing expression of NPC1L1 (Niemann-Pick disease, type C1, gene-like 1) and/or LIPG (Lipase, endothelial) mRNA.

### BACKGROUND ART

In recent years, with a change in Japanese dietary habits from Japanese-style food to Western-style food, Japanese people have taken in excessive energy (an increase in the intake of fat and sucrose). The lack of exercise, as well as the aforementioned intake of excessive energy, has resulted in an increase in lifestyle-related diseases such as obesity and diabetes. According to the reports by the Organization for Economic Cooperation and Development, an increase in obese people is not a problem characteristic for Japan, but a global-scale problem regarding public health, including the United States of America, European countries such as England and Austria, and other major countries such as China and India. It has been known that the life time of severe obese patients is shorter than that of healthy people with normal body weights by 8 to 10 years and that the annual medical costs of obese patients are higher than those of healthy people with normal body weights by 25% (Non Patent Literature 1). The latest data from the Centers for Disease Control and Prevention and the National Center for Health Statistics, U.S.A. have reported that “overweight” (BMI: less than 29.9) constitutes 58.0% of the adult population, “obesity” (BMI: 30-39.9) constitutes 35.7% thereof, and “excessive obesity” (BMI: 40.0 or greater) constitutes 6.3% thereof. Obesity is associated with an increase in the total amount of fat tissues (i.e. body fat), and in particular, fat tissues accumulated in abdomen, and it is a chronic disease directly causing metabolic vascular anomaly including hyperglycemia, hyperlipidemia, hypertension, fatty liver disease and various vascular disorders, and also causing many dangerous comorbidities such as inflammatory disease, progeria and several forms of cancers. Moreover, as a result of an increase in clinical grounds, the best method for regulating type 2 diabetes has been found to be weight reduction. Since suppression of obesity leads to prevention of various diseases that would be developed subsequently, it has been expected to establish an effective method for preventing obesity.

NPC1L1 (Niemann-Pick disease, type C1, gene-like 1) is expressed at a high level in lung, pancreas and liver (GenBank Accession No.: NM_{—}013389). The NPC1L1 protein encoded by this gene is an N-glycosylated protein containing a four-amino acid motif that functions as a trans-Golgi network for plasma membrane transport signal (Non Patent Literatures 2, 3, 4 and 5). The tissue distribution of such NPC1L1 protein is restricted, and this protein is abundantly present in the stomach and bowl. In addition, this protein has homology of 42% at the amino acid sequence level with human NPC1 (Non Patent Literature 6). The NPC1 protein has cholesterol transport ability. Thus, it has been known that if this protein were lost, Niemann-Pick disease would be developed, which is congenital lipid metabolism abnormality and causes accumulation of low-density lipoprotein (LDL)-derived non-esterified cholesterol in lysosome (Non Patent Literature 7). Moreover, it has also been reported that NPC1L1 is a protein that is a key for cholesterol absorption via small intestine and has an effect on a decrease in the LDL value in plasma, and that the NPC1L1 is also a target protein of ezetimibe, and the polymorphism of this gene leads to a difference in reactivity with ezetimibe (Non Patent Literature 8). Furthermore, NPC1L1 has been known to have various biological activities. It has been reported that, when NPC1L1-knockout mice were fed with high-fat diet without cholesterol, an increase in body weight was not observed in the knockout mice, in comparison with wild-type mice which had no change in eating and fat absorption via small intestine (Non Patent Literature 9). Accordingly, it is expected that NPC1L1 can be used to develop an effective anti-obesity agent based on the suppression of the expression of this gene.

LIPG (Lipase, endothelial) is a gene encoding endothelial lipase (EL), and it has been known that the polymorphism of this gene is associated with plasma HDL-C level (GenBank Accession No.: NM_{—}006033). It has also been known that endothelial lipase is an enzyme which HDL or other lipoproteins are hydroxylated with, and is widely distributed in a body (Non Patent Literatures 10 and 11). The mRNA level of endothelial lipase is accelerated in HUVEC and coronary artery endothelial cells by inflammatory cytokine assumed to be involved in vascular diseases or by blood vessel remodeling. In short, it has been suggested that endothelial lipase be complicatedly involved in lipid metabolism in blood vessels, and that such endothelial lipase be also involved in vascular diseases such as atheroma (Non Patent Literature 12). A method which utilizes the antisense of this gene as a therapeutic agent for atheroma has also been reported (Patent Literature 6). It has been found that, in mice in which endothelial lipase has been knocked out, the plasma HDL-C level increases, and the levels of apolipoprotein A-1 and E increase. Thus, with the results that human LIPG polymorphism correlates with HDL-C, it has been demonstrated that endothelial lipase is a principal factor regarding the concentration, form and metabolism of HDL in human (Non Patent Literatures 13 and 14). Endothelial lipase has been known to have various biological activities. It has been reported that the value of endothelial lipase in plasma positively correlates with HDL-C value, BMI, waist circumference, insulin resistance and inflammatory marker, and that it promotes metabolic syndrome (Non Patent Literature 15). In addition, in mice in which EL has been knocked out, the survival rate increases after injection of bacterial toxin, lipopolysaccharide (LPS), and thus, it is expected that anti-inflammatory action can be obtained by suppressing expression level of EL (Non Patent Literature 16).

Cholesteryl ester transfer protein (CETP) is an extremely highly hydrophobic protein which transfers cholesteryl ester from HDL cholesterol to LDL cholesterol, very low density lipoprotein (VLDL) cholesterol or the like. It is possible to increase HDL cholesterol by inhibiting such transfer by CETP. Hence, it is expected that a CETP inhibitor will be used as a therapeutic agent for diseases such as lipid disorder (hyperlipidemia), arteriosclerosis, atherosclerosis, peripheral vascular disease, hyper-LDL cholesterolemia, hypo-HDL cholesterolemia, hypercholesterolemia, hypertriglyceridemia, familial hypercholesterolemia, cardiovascular disturbance, angina pectoris, ischemia, cardiac ischemia, thrombosis, myocardial infarction, reperfusion injury, angioplasty restenosis, or hypertension. As compounds which inhibit such cholesteryl ester transfer protein (CETP) activity, Torcetrapib (Patent Literature 3), Anacetrapib (Patent Literature 4), Dalcetrapib (Patent Literature 5) and the like have been known, for example.

On the other hand, it has not yet been known that the CETP inhibitor is useful for suppressing expression of NPC1L1 or LIPG mRNA, or preventing and/or treating obesity.

### CITATION LIST

### Patent Literature

[Patent Literature 1] JP-A-2004-505637
[Patent Literature 2] International Publication No. WO2007/037299
[Patent Literature 3] International Publication No. WO2000/017164
[Patent Literature 4] International Publication No. WO2006/014357
[Patent Literature 5] International Publication No. WO1998/035937
[Patent Literature 6] International Publication No. WO2004/055162

### Non Patent Literature

[Non Patent Literature 1] OECD, the Organization for Economic Cooperation and Development, “Himan to Yobo no Keizaigaku (Economics for Obesity and Prevention Thereof): FIT, NOT FAT,” [online], Sep. 23, 2010, [searched on Mar. 18, 2011] Internet, <URL: http://www.oecdtokyo2.org/pdf/theme_pdf/health_pdf/201009 23fit.pdf>
[Non Patent Literature 2] EMBO J., 12: 2219-28 (1993)
[Non Patent Literature 3] J. Cell. Biol., 120: 1123-35 (1993)
[Non Patent Literature 4] J. Cell. Biol., 125: 253-268 (1994)
[Non Patent Literature 5] Science, 272: 227-34 (1996)
[Non Patent Literature 6] Science, 277: 228-31 (1997)
[Non Patent Literature 7] Genomics, 65 (2): 137 (2000)
[Non Patent Literature 8] J. Atheroscler. Thromb., 17 (4): 356 (2010)
[Non Patent Literature 9] J. Lipid Res., 51 (10) 3024-33 (2010)
[Non Patent Literature 10] J. Biol. Chem., 274, 14170 (1999)
[Non Patent Literature 11] Nat. Genet., 21, 424 (1999)
[Non Patent Literature 12] Biochem. Biophys. Res. Commun., 272, 90 (2000)
[Non Patent Literature 13] Proc Natl Acad Sci USA 2003, 100 (5): 2748
[Non Patent Literature 14] J Clin Invest 2003, 111 (3): 347
[Non Patent Literature 15] Atherosclerosis Supplement, Vol. 10, Issue 2, Abs: 612 (2009)

[Non Patent Literature 16] J. Lipid Res., Vol. 52 (1):57 (2011)

### SUMMARY OF INVENTION

### Technical Problem

It is an object of the present invention to provide an agent for preventing and/or treating obesity. In addition, it is another object of the present invention to provide an agent for suppressing expression of NPC1L1 mRNA and/or LIPG mRNA.

### Solution to Problem

As a result of intensive studies directed towards achieving the aforementioned objects, the present inventors have found that a compound represented by the following general formula (I) or general formula (II), or a salt thereof, or a solvate thereof, which has been known as an inhibitor of cholesteryl ester transfer protein (CETP), has an excellent action to suppress the expression of NPC1L1 and LIPG mRNAs, and that these substances also have action to suppress NPC1L1 and LIPG protein production. As a result, the inventors have found that these substances are also useful as agents for preventing and/or treating obesity, thereby completing the present invention. Specifically, the present invention includes the following examples [1] to [40].

[1] An agent for suppressing expression of NPC1L1 and/or LIPG mRNA, comprising, as an active ingredient, a compound having activity of inhibiting cholesteryl ester transfer protein, or a salt thereof, or a solvate thereof.
[2] The agent for suppressing expression according to [1] above, wherein the compound having activity of inhibiting cholesteryl ester transfer protein, or a salt thereof, or a solvate thereof is a compound represented by the following formula (I) or (II), or a salt thereof, or a solvate thereof:

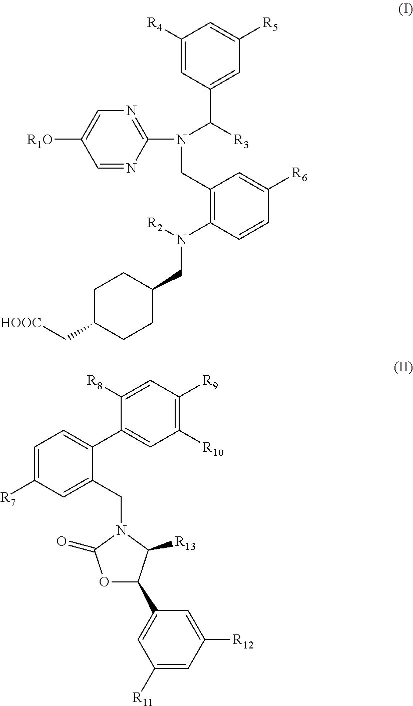

wherein, in the formula (I),
R₁ represents a C₁₋₆ alkylthio C₁₋₆ alkyl group, a C₁₋₆ alkylsulfinyl C₁₋₆ alkyl group or a C₁₋₆ alkylsulfonyl C₁₋₆ alkyl group,
R₂ represents a hydrogen atom or an optionally halogen atom-substituted C₁₋₆ alkyl group,
R₃ represents an optionally halogen atom-substituted C₁₋₆ alkyl group,
R₄ and R₅, which are the same or different, each represent a hydrogen atom, a halogen atom, an optionally halogen atom-substituted C₁₋₆ alkyl group, an optionally halogen atom-substituted C₁₋₆ alkoxy group, or a cyano group, and
R₆ represents a hydrogen atom, a halogen atom, an optionally halogen atom-substituted C₁₋₆ alkyl group, or an optionally halogen atom-substituted C₁₋₆ alkoxy group, and
in the formula (II),
R₇, R₈, R₉, R₁₀, R₁₁, R₁₂ and R₁₃, which are the same or different, each represent a hydrogen atom, a hydroxy group, a halogen atom, an optionally halogen atom-substituted C₁₋₆ alkyl group, or an optionally halogen atom-substituted C₁₋₆ alkoxy group.
[3] The agent for suppressing expression according to [1] above, comprising, as an active ingredient, at least one compound selected from the group consisting of the following compounds, or a salt thereof, or a solvate thereof: trans-{4-[({2-[({1-[3,5-bis(trifluoromethyl)phenyl]ethyl}{5-[2-(methylsulfonyl)ethoxy]pyrimidin-2-yl}amino)methyl]-4-(trifluoromethyl)phenyl}(ethyl)amino)methyl]cyclohexyl}ac etic acid, (S)-(−)-trans-{4-[({2-[({1-[3,5-bis(trifluoromethyl)phenyl]ethyl}{5-[2-(methylsulfonyl)ethoxy]pyrimidin-2-yl}amino)methyl]-4-(trifluoromethyl)phenyl}(ethyl)amino)methyl]cyclohexyl}ac etic acid, and (R)-(+)-trans-{4-[({2-[({1-[3,5-bis(trifluoromethyl)phenyl]ethyl}{5-[2-(methylsulfonyl)ethoxy]pyrimidin-2-yl}amino)methyl]-4-(trifluoromethyl)phenyl}(ethyl)amino)methyl]cyclohexyl}ac etic acid.
[4] The agent for suppressing expression according to [1] above, comprising, as an active ingredient, (4S,5R)-5-[3,5-bis(trifluoromethyl)phenyl]-3-((4′-fluoro-5′-isopropyl-2′-methoxy-4-(trifluoromethyl)-[1,1′-biphenyl]-2-yl)methyl)-4-methyloxazolidin-2-one, or a salt thereof, or a solvate thereof.
[5] An agent for suppressing NPC1L1 and/or LIPG protein production, comprising, as an active ingredient, a compound having activity of inhibiting cholesteryl ester transfer protein, or a salt thereof, or a solvate thereof.
[6] The agent for suppressing protein production according to [5] above, wherein the compound having activity of inhibiting cholesteryl ester transfer protein, or a salt thereof, or a solvate thereof is the compound represented by the above formula (I) or (II), or a salt thereof, or a solvate thereof.
[7] The agent for suppressing protein production according to [5] above, comprising, as an active ingredient, at least one compound selected from the group consisting of the following compounds, or a salt thereof, or a solvate thereof: trans-{4-[({2-[({1-[3,5-bis(trifluoromethyl)phenyl]ethyl}{5-[2-(methylsulfonyl)ethoxy]pyrimidin-2-yl}amino)methyl]-4-(trifluoromethyl)phenyl}(ethyl)amino)methyl]cyclohexyl}ac etic acid, (S)-(−)-trans-{4-[({2-[({1-[3,5-bis(trifluoromethyl)phenyl]ethyl}{5-[2-(methylsulfonyl)ethoxy]pyrimidin-2-yl}amino)methyl]-4-(trifluoromethyl)phenyl}(ethyl)amino)methyl]cyclohexyl}ac etic acid, and (R)-(+)-trans-{4-[({2-[({1-[3,5-bis(trifluoromethyl)phenyl]ethyl)}{5-[2-(methylsulfonyl)ethoxy]pyrimidin-2-yl}amino)methyl]-4-(trifluoromethyl)phenyl}(ethyl)amino)methyl]cyclohexyl}ac etic acid.
[8] The agent for suppressing protein production according to [5] above, comprising, as an active ingredient, (4S,5R)-5-[3,5-bis(trifluoromethyl)phenyl]-3-((4′-fluoro-5′-isopropyl-2′-methoxy-4-(trifluoromethyl)-[1,1′-biphenyl]-2-yl)methyl)-4-methyloxazolidin-2-one, or a salt thereof, or a solvate thereof.
[9] An agent for preventing and/or treating obesity, comprising, as an active ingredient, a compound represented by the following formula (I), or a salt thereof, or a solvate thereof:

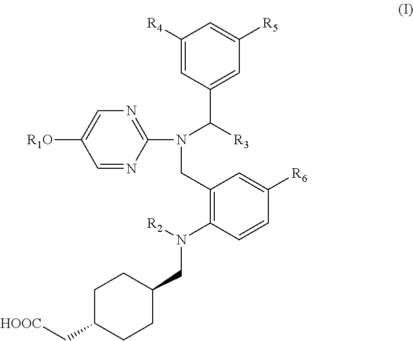

wherein R₁ to R₆ are the same as those described above.
[10] The agent for preventing and/or treating obesity according to [9] above, comprising, as an active ingredient, at least one compound selected from the group consisting of the following compounds, or a salt thereof, or a solvate thereof: trans-{4-[({2-[({1-[3,5-bis(trifluoromethyl)phenyl]ethyl}{5-[2-(methylsulfonyl)ethoxy]pyrimidin-2-yl}amino)methyl]-4-(trifluoromethyl)phenyl}(ethyl)amino)methyl]cyclohexyl}ac etic acid, (S)-(−)-trans-{4-[({2-[({1-[3,5-bis(trifluoromethyl)phenyl]ethyl}{5-[2-(methylsulfonyl)ethoxy]pyrimidin-2-yl}amino)methyl]-4-(trifluoromethyl)phenyl}(ethyl)amino)methyl]cyclohexyl}ac etic acid, and (R)-(+)-trans-{4-[({2-[({1-[3,5-bis(trifluoromethyl)phenyl]ethyl}{5-[2-(methylsulfonyl)ethoxy]pyrimidin-2-yl}amino)methyl]-4-(trifluoromethyl)phenyl}(ethyl)amino)methyl]cyclohexyl}ac etic acid.
[11] A compound having activity of inhibiting cholesteryl ester transfer protein, or a salt thereof, or a solvate thereof, for suppressing expression of NPC1L1 and/or LIPG mRNA.
[12] The compound according to [11] above, or a salt thereof, or a solvate thereof, wherein the compound having activity of inhibiting cholesteryl ester transfer protein, or a salt thereof, or a solvate thereof is the compound represented by the above formula (I) or (II), or a salt thereof, or a solvate thereof.
[13] The compound according to [11] above, or a salt thereof, or a solvate thereof, wherein the compound having activity of inhibiting cholesteryl ester transfer protein is at least one compound selected from the group consisting of: trans-{4-[({2-[({1-[3,5-bis(trifluoromethyl)phenyl]ethyl}{5-[2-(methylsulfonyl)ethoxy]pyrimidin-2-yl}amino)methyl]-4-(trifluoromethyl)phenyl}(ethyl)amino)methyl]cyclohexyl}ac etic acid, (S)-(−)-trans-{4-[({2-[({1-[3,5-bis(trifluoromethyl)phenyl]ethyl}{5-[2-(methylsulfonyl)ethoxy]pyrimidin-2-yl}amino)methyl]-4-(trifluoromethyl)phenyl}(ethyl)amino)methyl]cyclohexyl}ac etic acid, and (R)-(+)-trans-{4-[({2-[({1-[3,5-bis(trifluoromethyl)phenyl]ethyl}{5-[2-(methylsulfonyl)ethoxy]pyrimidin-2-yl}amino)methyl]-4-(trifluoromethyl)phenyl}(ethyl)amino)methyl]cyclohexyl}ac etic acid.
[14] The compound according to [11] above, or a salt thereof, or a solvate thereof, wherein the compound having activity of inhibiting cholesteryl ester transfer protein is (4S,5R)-5-[3,5-bis(trifluoromethyl)phenyl]-3-((4′-fluoro-5′-isopropyl-2′-methoxy-4-(trifluoromethyl)-[1,1′-biphenyl]-2-yl)methyl)-4-methyloxazolidin-2-one.
[15] A compound having activity of inhibiting cholesteryl ester transfer protein, or a salt thereof, or a solvate thereof, for suppressing NPC1L1 and/or LIPG protein production.
[16] The compound according to [15] above, or a salt thereof, or a solvate thereof, wherein the compound having activity of inhibiting cholesteryl ester transfer protein, or a salt thereof, or a solvate thereof is the compound represented by the above formula (I) or (II), or a salt thereof, or a solvate thereof.
[17] The compound according to [15] above, or a salt thereof, or a solvate thereof, wherein the compound having activity of inhibiting cholesteryl ester transfer protein is at least one compound selected from the group consisting of: trans-{4-[({2-[({1-[3,5-bis(trifluoromethyl)phenyl]ethyl}{5-[2-(methylsulfonyl)ethoxy]pyrimidin-2-yl}amino)methyl]-4-(trifluoromethyl)phenyl}ethyl)amino)methyl]cyclohexyl}ac etic acid, (S)-(−)-trans-{4-[({2-[({1-[3,5-bis(trifluoromethyl)phenyl]ethyl}{5-[2-(methylsulfonyl)ethoxy]pyrimidin-2-yl}amino)methyl]-4-(trifluoromethyl)phenyl}(ethyl)amino)methyl]cyclohexyl}-acetic acid, and (R)-(+)-trans-{4-[({2-[({1-[3,5-bis(trifluoromethyl)phenyl]ethyl}{5-[2-(methylsulfonyl)ethoxy]pyrimidin-2-yl}amino)methyl]-4-(trifluoromethyl)phenyl}(ethyl)amino)methyl]cyclohexyl}ac etic acid.
[18] The compound according to [15] above, or a salt thereof, or a solvate thereof, wherein the compound having activity of inhibiting cholesteryl ester transfer protein is (4S,5R)-5-[3,5-bis(trifluoromethyl)phenyl]-3-((4′-fluoro-5′-isopropyl-2′-methoxy-4-(trifluoromethyl)-[1,1′-biphenyl]-2-yl)methyl)-4-methyloxazolidin-2-one.
[19] The compound represented by the above formula (I), or a salt thereof, or a solvate thereof, for preventing and/or treating obesity.
[20] The compound according to [19] above, or a salt thereof, or a solvate thereof, wherein the compound having activity of inhibiting cholesteryl ester transfer protein is at least one compound selected from the group consisting of the following compounds, or a salt thereof, or a solvate thereof: trans-{4-[({2-[({1-[3,5-bis(trifluoromethyl)phenyl]ethyl}{5-[2-(methylsulfonyl)ethoxy]pyrimidin-2-yl}amino)methyl]-4-(trifluoromethyl)phenyl}(ethyl)amino)methyl]cyclohexyl}ac etic acid, (S)-(−)-trans-{4-[({2-[({1-[3,5-bis(trifluoromethyl)phenyl]ethyl}{5-[2-(methylsulfonyl)ethoxy]pyrimidin-2-yl}amino)methyl]-4-(trifluoromethyl)phenyl}(ethyl)amino)methyl]cyclohexyl}ac etic acid, and (R)-(+)-trans-{4-[({2-[({1-[3,5-bis(trifluoromethyl)phenyl]ethyl}{5-[2-(methylsulfonyl)ethoxy]pyrimidin-2-yl}amino)methyl]-4-(trifluoromethyl)phenyl}(ethyl)amino)methyl]cyclohexyl}ac etic acid.
[21] Use of a compound having activity of inhibiting cholesteryl ester transfer protein, or a salt thereof, or a solvate thereof, for manufacturing an agent for suppressing expression of NPC1L1 and/or LIPG mRNA.
[22] The use according to [21] above, wherein the compound having activity of inhibiting cholesteryl ester transfer protein, or a salt thereof, or a solvate thereof is the compound represented by the above formula (I) or (II), or a salt thereof, or a solvate thereof.
[23] The use according to [21] above, wherein the compound having activity of inhibiting cholesteryl ester transfer protein is at least one compound selected from the group consisting of: trans-{4-[({2-[({1-[3,5-bis(trifluoromethyl)phenyl]ethyl}{5-[2-(methylsulfonyl)ethoxy]pyrimidin-2-yl}amino)methyl]-4-(trifluoromethyl)phenyl}(ethyl)amino)methyl]cyclohexyl}ac etic acid, (S)-(−)-trans-{4-[({2-[({1-[3,5-bis(trifluoromethyl)phenyl]ethyl}{5-[2-(methylsulfonyl)ethoxy]pyrimidin-2-yl}amino)methyl]-4-(trifluoromethyl)phenyl}(ethyl)amino)methyl]cyclohexyl}ac etic acid, and (R)-(+)-trans-{4-[({2-[({1-[3,5-bis(trifluoromethyl)phenyl]ethyl}{5-[2-(methylsulfonyl)ethoxy]pyrimidin-2-yl}amino)methyl]-4-(trifluoromethyl)phenyl}(ethyl)amino)methyl]cyclohexyl}ac etic acid.
[24] The use according to [21] above, wherein the compound having activity of inhibiting cholesteryl ester transfer protein is (4S,5R)-5-[3,5-bis(trifluoromethyl)phenyl]-3-((4′-fluoro-5′-isopropyl-2′-methoxy-4-(trifluoromethyl)-[1,1′-biphenyl]-2-yl)methyl)-4-methyloxazolidin-2-one.
[25] Use of a compound having activity of inhibiting cholesteryl ester transfer protein, or a salt thereof, or a solvate thereof, for manufacturing an agent for suppressing NPC1L1 and/or LIPG protein production.
[26] The use according to [25] above, wherein the compound having activity of inhibiting cholesteryl ester transfer protein, or a salt thereof, or a solvate thereof is the compound represented by the above formula (I) or (II), or a salt thereof, or a solvate thereof.
[27] The use according to [25] above, wherein the compound having activity of inhibiting cholesteryl ester transfer protein is at least one compound selected from the group consisting of: trans-{4-[({2-[({1-[3,5-bis(trifluoromethyl)phenyl]ethyl}{5-[2-(methylsulfonyl)ethoxy]pyrimidin-2-yl}amino)methyl]-4-(trifluoromethyl)phenyl}(ethyl)amino)methyl]cyclohexyl}ac etic acid, (S)-(−)-trans-{4-[({2-[({1-[3,5-bis(trifluoromethyl)phenyl]ethyl}{5-[2-(methylsulfonyl)ethoxy]pyrimidin-2-yl}amino)methyl]-4-(trifluoromethyl)phenyl}(ethyl)amino)methyl]cyclohexyl}ac etic acid, and (R)-(+)-trans-{4-[({2-[({1-[3,5-bis(trifluoromethyl)phenyl]ethyl}{5-[2-(methylsulfonyl)ethoxy]pyrimidin-2-yl}amino)methyl]-4-(trifluoromethyl)phenyl}(ethyl)amino)methyl]cyclohexyl}ac etic acid.
[28] The use according to [25] above, wherein the compound having activity of inhibiting cholesteryl ester transfer protein is (4S,5R)-5-[3,5-bis(trifluoromethyl)phenyl]-3-((4′-fluoro-5′-isopropyl-2′-methoxy-4-(trifluoromethyl)-[1,1′-biphenyl]-2-yl)methyl)-4-methyloxazolidin-2-one.
[29] Use of the compound represented by the above formula (I), or a salt thereof, or a solvate thereof, for manufacturing an agent for preventing and/or treating obesity.
[30] The use according to [29] above, wherein the compound represented by the formula (I) is at least one compound selected from the group consisting of: trans-{4-[({2-[({1-[3,5-bis(trifluoromethyl)phenyl]ethyl}{5-[2-(methylsulfonyl)ethoxy]pyrimidin-2-yl}amino)methyl]-4-(trifluoromethyl)phenyl}(ethyl)amino)methyl]cyclohexyl}ac etic acid, (S)-(−)-trans-{4-[({2-[({1-[3,5-bis(trifluoromethyl)phenyl]ethyl}{5-[2-(methylsulfonyl)ethoxy]pyrimidin-2-yl}amino)methyl]-4-(trifluoromethyl)phenyl}(ethyl)amino)methyl]cyclohexyl}ac etic acid, and (R)-(+)-trans-{4-[({2-[({1-[3,5-bis(trifluoromethyl)phenyl]ethyl}{5-[2-(methylsulfonyl)ethoxy]pyrimidin-2-yl}amino)methyl]-4-(trifluoromethyl)phenyl}(ethyl)amino)methyl]cyclohexyl}ac etic acid.
[31] A method for suppressing expression of NPC1L1 and/or LIPG mRNA, comprising administering a compound having activity of inhibiting cholesteryl ester transfer protein, or a salt thereof, or a solvate thereof.
[32] The method for suppressing expression according to [31] above, wherein the compound having activity of inhibiting cholesteryl ester transfer protein, or a salt thereof, or a solvate thereof is a compound represented by the above formula (I) or (II), or a salt thereof, or a solvate thereof.
[33] The method for suppressing expression according to [31] above, wherein the method comprises administering at least one compound selected from the group consisting of the following compounds, or a salt thereof, or a solvate thereof: trans-{4-[({2-[({1-[3,5-bis(trifluoromethyl)phenyl]ethyl}{5-[2-(methylsulfonyl)ethoxy]pyrimidin-2-yl}amino)methyl]-4-(trifluoromethyl)phenyl}(ethyl)amino)methyl]cyclohexyl}ac etic acid, (S)-(−)-trans-{4-[({2-[({1-[3,5-bis(trifluoromethyl)phenyl]ethyl}{5-[2-(methylsulfonyl)ethoxy]pyrimidin-2-yl}amino)methyl]-4-(trifluoromethyl)phenyl}(ethyl)amino)methyl]cyclohexyl}ac etic acid, and (R)-(+)-trans-{4-[({2-[({1-[3,5-bis(trifluoromethyl)phenyl]ethyl}{5-[2-(methylsulfonyl)ethoxy]pyrimidin-2-yl}amino)methyl]-4-(trifluoromethyl)phenyl}(ethyl)amino)methyl]cyclohexyl}ac etic acid.
[34] The method for suppressing expression according to [31] above, wherein the method comprises administering (4S,5R)-5-[3,5-bis(trifluoromethyl)phenyl]-3-((4′-fluoro-5′-isopropyl-2′-methoxy-4-(trifluoromethyl)-[1,1′-biphenyl]-2-yl)methyl)-4-methyloxazolidin-2-one, or a salt thereof, or a solvate thereof.
[35] A method for suppressing NPC1L1 and/or LIPG protein production, comprising administering a compound having activity of inhibiting cholesteryl ester transfer protein, or a salt thereof, or a solvate thereof.
[36] The method for suppressing expression according to [35] above, wherein the compound having activity of inhibiting cholesteryl ester transfer protein, or a salt thereof, or a solvate thereof is a compound represented by the above formula (I) or (II), or a salt thereof, or a solvate thereof.
[37] The method for suppressing protein production according to [35] above, wherein the method comprises administering at least one compound selected from the group consisting of the following compounds, or a salt thereof, or a solvate thereof: trans-{4-[({2-[({1-[3,5-bis(trifluoromethyl)phenyl]ethyl}{5-[2-(methylsulfonyl)ethoxy]pyrimidin-2-yl}amino)methyl]-4-(trifluoromethyl)phenyl}(ethyl)amino)methyl]cyclohexyl}ac etic acid, (S)-(−)-trans-{4-[({2-[({1-[3,5-bis(trifluoromethyl)phenyl]ethyl}{5-[2-(methylsulfonyl)ethoxy]pyrimidin-2-yl}amino)methyl]-4-(trifluoromethyl)phenyl}(ethyl)amino)methyl]cyclohexyl}ac etic acid, and (R)-(+)-trans-{4-[({2-[({1-[3,5-bis(trifluoromethyl)phenyl]ethyl}{5-[2-(methylsulfonyl)ethoxy]pyrimidin-2-yl}amino)methyl]-4-(trifluoromethyl)phenyl}(ethyl)amino)methyl]cyclohexyl}ac etic acid.
[38] The method for suppressing protein production according to [35] above, wherein the method comprises administering (4S,5R)-5-[3,5-bis(trifluoromethyl)phenyl]-3-((4′-fluoro-5′-isopropyl-2′-methoxy-4-(trifluoromethyl)-[1,1′-biphenyl]-2-yl)methyl)-4-methyloxazolidin-2-one, or a salt thereof, or a solvate thereof.
[39] A method for preventing and/or treating obesity, comprising administering the compound represented by the above formula (I), or a salt thereof, or a solvate thereof.
[40] The method for preventing and/or treating obesity according to [39] above, wherein the method comprises administering at least one compound selected from the group consisting of the following compounds, or a salt thereof, or a solvate thereof: trans-{4-[({2-[({1-[3,5-bis(trifluoromethyl)phenyl]ethyl}{5-[2-(methylsulfonyl)ethoxy]pyrimidin-2-yl}amino)methyl]-4-(trifluoromethyl)phenyl}(ethyl)amino)methyl]cyclohexyl}ac etic acid, (S)-(−)-trans-{4-[({2-[({1-[3,5-bis(trifluoromethyl)phenyl]ethyl}{5-[2-(methylsulfonyl)ethoxy]pyrimidin-2-yl}amino)methyl]-4-(trifluoromethyl)phenyl}(ethyl)amino)methyl]cyclohexyl}ac etic acid, and (R)-(+)-trans-{4-[({2-[({1-[3,5-bis(trifluoromethyl)phenyl]ethyl}{5-[2-(methylsulfonyl)ethoxy]pyrimidin-2-yl}amino)methyl]-4-(trifluoromethyl)phenyl}(ethyl)amino)methyl]cyclohexyl}ac etic acid.

The compound (III), trans-{4-[({2-[({1-[3,5-bis(trifluoromethyl)phenyl]ethyl}{5-[2-(methylsulfonyl)ethoxy]pyrimidin-2-yl}amino)methyl]-4-(trifluoromethyl)phenyl}(ethyl)amino)methyl]cyclohexyl}ac etic acid, and the compound (IV), (4S,5R)-5-[3,5-bis(trifluoromethyl)phenyl]-3-((4′-fluoro-5′-isopropyl-2′-methoxy-4-(trifluoromethyl)-[1,1′-biphenyl]-2-yl)methyl)-4-methyloxazolidin-2-one, are more preferable as specific compounds represented by the general formula (I) or (II).

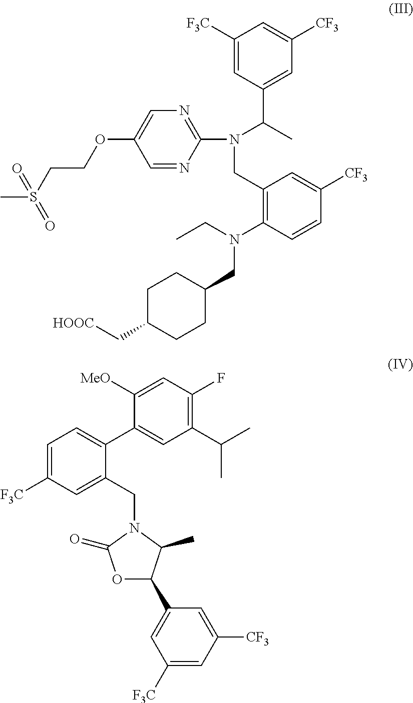

### Advantageous Effects of Invention

As specifically described in the after-mentioned Examples, the compounds having CETP inhibitory activity according to the present invention have a strong action to suppress the expression of NPC1L1 and LIPG mRNAs. In addition, since these compounds have an action to suppress NPC1L1 and LIPG protein production, they can be effectively used for preventing and/or treating obesity.

### BRIEF DESCRIPTION OF DRAWINGS

FIG. 1 is a view showing the relative LIPG mRNA expression levels in cells, to which Compound 2 and Anacetrapib were each added.

FIG. 2 is a view showing the relative NPC1L1 mRNA expression levels in cells, to which Compound 2 and Anacetrapib were each added.

### DESCRIPTION OF EMBODIMENTS

The active ingredient of the pharmaceutical of the present invention is a compound having activity of inhibiting CETP. Examples of such a compound include the compounds described in Patent Literatures 3 to 5, such as the above-mentioned Torcetrapib, Anacetrapib and Dalcetrapib, and the compounds described in WO99/041237, WO00/018721, WO05/097805, WO06/073973, WO08/079,427, WO08/129,951, WO08/156,718, WO09/007,259, Bioorganic & Medicinal Chemistry Letters, 6, 1951-1954 (1996), Bioorganic & Medicinal Chemistry Letters, 15, 3611-3614 (2005), Bioorganic & Medicinal Chemistry Letters, 17, 2608-2613 (2007), etc. Preferred examples of the aforementioned compound include the compound represented by the general formula (I) or (II), or a salt thereof, or a solvate thereof.

The compound represented by the general formula (I) is described in WO08/129,951 (Compound 1 in Example 45), and it can be produced by the method described in the same publication as described above. In addition, the compound represented by the general formula (II) and including Anacetrapib is described in WO06/014357 (Anacetrapib in Example 73), and it can be produced by the method described in the same publication as described above.

In the present description, the “C₁₋₆ alkyl” in the phrase “a C₁₋₆ alkylthio C₁₋₆ alkyl group, a C₁₋₆ alkylsulfinyl C₁₋₆ alkyl group or a C₁₋₆ alkylsulfonyl C₁₋₆ alkyl group” means a linear or branched alkyl having 1 to 6 carbon atoms. Such C₁₋₆ alkyl is preferably “C₁₋₃ alkyl” and more preferably “methyl or ethyl”. Examples of the “C₁-C₆ alkyl” include methyl, ethyl, n-propyl, isopropyl, n-butyl, isobutyl, sec-butyl, tert-butyl, n-pentyl, isopentyl, neopentyl, 2-methylbutyl, 1-ethylpropyl, n-hexyl, isohexyl, 3-methylpentyl, 2-methylpentyl, 1-methylpentyl, 3,3-dimethylbutyl, 2,2-dimethylbutyl, 1,1-dimethylbutyl, 1,2-dimethylbutyl, 1,3-dimethylbutyl, 2,3-dimethylbutyl, 1-ethylbutyl, and 2-ethylbutyl.

In the present description, examples of the “C₁-C₆ alkylthio” in the phrase “C₁₋₆ alkylthio C₁₋₆ alkyl group” include methylthio, ethylthio, n-propylthio, isopropylthio, n-butylthio, isobutylthio, sec-butylthio, tert-butylthio, n-pentylthio, isopentylthio, neopentylthio, 2-methylbutylthio, 1-ethylpropylthio, n-hexylthio, isohexylthio, 3-methylpentylthio, 2-methylpentylthio, 1-methylpentylthio, 3,3-dimethylbutylthio, 2,2-dimethylbutylthio, 1,1-dimethylbutylthio, 1,2-dimethylbutylthio, 1,3-dimethylbutylthio, 2,3-dimethylbutylthio, 1-ethylbutylthio, and 2-ethylbutylthio.

In the present description, examples of the “C₁-C₆ alkylsulfinyl” in the phrase “C₁₋₆ alkylsulfinyl C₁₋₆ alkyl group” include methylsulfinyl, ethylsulfinyl, n-propylsulfinyl, isopropylsulfinyl, n-butylsulfinyl, isobutylsulfinyl, sec-butylsulfinyl, tert-butylsulfinyl, n-pentylsulfinyl, isopentylsulfinyl, neopentylsulfinyl, 2-methylbutylsulfinyl, 1-ethylpropylsulfinyl, n-hexylsulfinyl, isohexylsulfinyl, 3-methylpentylsulfinyl, 2-methylpentylsulfinyl, 1-methylpentylsulfinyl, 3,3-dimethylbutylsulfinyl, 2,2-dimethylbutylsulfinyl, 1,1-dimethylbutylsulfinyl, 1,2-dimethylbutylsulfinyl, 1,3-dimethylbutylsulfinyl, 2,3-dimethylbutylsulfinyl, 1-ethylbutylsulfinyl, and 2-ethylbutylsulfinyl.

In the present description, examples of the “C₁-C₆ alkylsulfonyl” in the phrase “C₁₋₆ alkylsulfonyl C₁₋₆ alkyl group” include methylsulfonyl, ethylsulfonyl, n-propylsulfonyl, isopropylsulfonyl, n-butylsulfonyl, isobutylsulfonyl, sec-butylsulfonyl, tert-butylsulfonyl, n-pentylsulfonyl, isopentylsulfonyl, neopentylsulfonyl, 2-methylbutylsulfonyl, 1-ethylpropylsulfonyl, n-hexylsulfonyl, isohexylsulfonyl, 3-methylpentylsulfonyl, 2-methylpentylsulfonyl, 1-methylpentylsulfonyl, 3,3-dimethylbutylsulfonyl, 2,2-dimethylbutylsulfonyl, 1,1-dimethylbutylsulfonyl, 1,2-dimethylbutylsulfonyl, 1,3-dimethylbutylsulfonyl, 2,3-dimethylbutylsulfonyl, 1-ethylbutylsulfonyl, and 2-ethylbutylsulfonyl.

In the present description, examples of the “halogen atom” include fluorine, chlorine, bromine, and iodine.

In the present description, the “optionally halogen atom-substituted C₁₋₆ alkyl group” means a linear or branched alkyl group having 1 to 6 carbon atoms, which is optionally substituted with a halogen atom. Examples of the optionally halogen atom-substituted C₁₋₆ alkyl group include methyl, ethyl, n-propyl, isopropyl, n-butyl, isobutyl, sec-butyl, tert-butyl, n-pentyl, isopentyl, neopentyl, 2-methylbutyl, 1-ethylpropyl, n-hexyl, isohexyl, trifluoromethyl, trichloromethyl, and trifluoroethyl. It is preferably an “optionally halogen atom-substituted C₁₋₃ alkyl group”, and more preferably an “optionally halogen atom-substituted methyl or ethyl group”.

In the present description, the “optionally halogen atom-substituted C₁₋₆ alkoxy group” means a linear or branched alkoxy group having 1 to 6 carbon atoms, which is optionally substituted with a halogen atom. Examples of the optionally halogen atom-substituted C₁₋₆ alkoxy group include methoxy, ethoxy, propoxy, isopropoxy, butoxy, isobutoxy, sec-butoxy, pentyloxy, hexyloxy, trifluoromethoxy, and trifluoroethoxy. It is preferably an “optionally halogen atom-substituted C₁₋₃ alkoxy group”, and more preferably an “optionally halogen atom-substituted methoxy or ethoxy group”.

As compounds represented by the general formula (I) or (II), any given stereoisomers or any given mixtures thereof, such as optically pure isomers or any given mixtures of the aforementioned isomers, racemic bodies, and any given geometric isomers or any given mixtures of the geometric isomers, may be used.

Examples of the stereoisomers of the compound represented by the general formula (I) include compounds represented by the following general formulae (I-1) and (I-2):

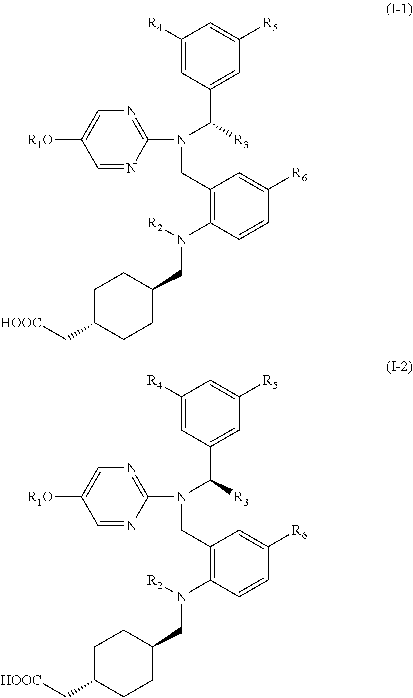

wherein R₁, R₂, R₃, R₄, R₅ and R₆ are the same as those described above.

The compound represented by the general formula (I) or (II) includes all of compounds which are metabolized in vivo and are converted to the compound represented by the general formula (I) or (II), namely, prodrugs. Examples of a group capable of forming a prodrug of the compound represented by the general formula (I) or (II) include the groups described in “Progress in Medicine,” Lifescience Medica, 1985, Vol. 5, pp. 2157-2161, and the groups described in “Iyakuhin no Kaihatsu (Development of Medicines)” published by Hirokawa Shoten, 1990, Vol. 7, Bunshi Sekkei (Molecular Design), pp. 163-198.

Examples of the preferred compound in the present invention include:
trans-{4-[({2-[({1-[3,5-bis(trifluoromethyl)phenyl]ethyl}{5-[2-(methylsulfonyl)ethoxy]pyrimidin-2-yl}amino)methyl]-4-(trifluoromethyl)phenyl}(ethyl)amino)methyl]cyclohexyl}ac etic acid (Compound 1); (S)-(−)-trans-{4-[({2-[({1-[3,5-bis(trifluoromethyl)phenyl]ethyl}{5-[2-(methylsulfonyl)ethoxy]pyrimidin-2-yl}amino)methyl]-4-(trifluoromethyl)phenyl}(ethyl)amino)methyl]cyclohexyl}ac etic acid (Compound 2);
(R)-(+)-trans-{4-[({2-[({1-[3,5-bis(trifluoromethyl)phenyl]ethyl}{5-[2-(methylsulfonyl)ethoxy]pyrimidin-2-yl}amino)methyl]-4-(trifluoromethyl)phenyl}(ethyl)amino)methyl]cyclohexyl}ac etic acid (Compound 3); and
(4S,5R)-5-[3,5-bis(trifluoromethyl)phenyl]-3-((4′-fluoro-5′-isopropyl-2′-methoxy-4-(trifluoromethyl)-[1,1′-biphenyl]-2-yl)methyl)-4-methyloxazolidin-2-one (Compound 4). However, the scope of the present invention is not limited to these compounds.

Examples of a salt of the compound represented by the general formula (I) or (II) include acid-added salts and base-added salts. The salt is not particularly limited, as long as it is a pharmaceutically acceptable salt. Examples of the acid-added salts include: acid-added salts with inorganic acids, such as hydrochloride, hydrobromide, hydroiodide, sulfate, nitrate or phosphate; and acid-added salts with organic acids, such as benzoate, methanesulfonate, ethanesulfonate, benzenesulfonate, p-toluenesulfonate, maleate, fumarate, tartrate, citrate, or acetate. Examples of the base-added salts include: base-added salts with metals, such as sodium salts, potassium salts, lithium salts, calcium salts, or magnesium salts; amine salts such as ammonia, trimethylamine, triethylamine, pyridine, collidine, or lutidine; and base-added salts with organic bases such as lysine, or arginine.

Examples of a solvent used to form a solvate of the compound represented by the general formula (I) or (II) or a salt thereof include water and physiologically acceptable organic solvents such as ethanol, hexane or ethyl acetate, but the examples are not limited thereto. An example of the active ingredient of the pharmaceutical of the present invention is a hydrate, but the examples are not limited thereto.

The compound represented by the general formula (I-1) (e.g. Compound 2) or the general formula (I-2) (e.g. Compound 3), which is an enantiomer of the compound represented by the general formula (I) (e.g. Compound 1), can be produced from the compound represented by the general formula (I) (e.g. Compound 1) by a method using a chiral column, or from a derivative of the compound represented by the general formula (I) (e.g. Compound 1) by a method fractionation according to preferential crystallization or the like and then induction to the compound represented by the general formula (I-1) (e.g. Compound 2) or the general formula (I-2) (e.g. Compound 3), etc.

The pharmaceutical of the present invention includes, as an active ingredient, the above described compound represented by the general formula (I), or a salt thereof, or a solvate thereof. The aforementioned active ingredient may directly be administered as the pharmaceutical of the present invention. However, preferably, the active ingredient may be prepared into and administered as a pharmaceutical composition for oral administration or parenteral administration, which can be produced according to a method well known to a person skilled in the art. Examples of a pharmaceutical composition suitable for oral administration include a tablet, a capsule, a powder, a fine granule, a granule, a liquid, and syrup. Examples of a pharmaceutical composition suitable for parenteral administration include injections such as intravenous injection or intramuscular injection, drops, suppository, inhalant, eye drops, nasal drops, a transdermal absorption agent and a transmucosal absorption agent, but the examples are not limited thereto.

The above described pharmaceutical composition can be produced by adding pharmacologically or pharmaceutically acceptable additives. Examples of such pharmacologically or pharmaceutically acceptable additives include an excipient, a binder, a thickener, a disintegrator, a surfactant, a lubricant, a dispersant, a buffer, a preservative, a corrigent, a perfume, a coating agent and a diluent, but the examples are not limited thereto.

The dosage of the pharmaceutical of the present invention is not particularly limited, and it can be selected, as appropriate, depending on the type of disease, preventive or therapeutic purpose, the type of an active ingredient, etc. Further, the dosage can be increased or decreased, as appropriate, depending on generally considerable various factors, such as the body weight and age of a patient, symptoms, and administration route. For example, in the case of oral administration, the compound according to the present invention can be used in the weight range of approximately 0.1 to 500 mg per adult per day. However, such dosage can be appropriately selected by a person skilled in the art, and it is not limited to the aforementioned range.

### EXAMPLES

Hereinafter, the present invention will be further described in the following Examples. However, these examples are not intended to limit the scope of the present invention. It is to be noted that abbreviations used in the below-mentioned Examples have the following meanings.

s: Singlet

d: Doublet

t: Triplet

q: Quartet

m: Multiplet

br: Broad

J: Coupling constant

Hz: Hertz

CDCl₃: Deuterated chloroform

¹H-NMR: Proton nuclear magnetic resonance

### Production Example 1

A compound produced according to the method disclosed in Example 45 of International Publication No. WO2008/129951 was used as Compound 1. In addition, as Compound 2 and Compound 3 which are both enantiomers of the Compound 1, those isolated from the Compound 1 by fractionation using a chiral column under the following conditions were used.

### Column: CHIRALCEL OD-H (4.6×250 mm)

Flow rate: 1.0 mL/min

### Detector: UV 242 nm

### Temp.: 40° C.

Mobile phase: hexane/EtOH/TFA=90/10/0.1
Retention time: (R)-(+)-form: 21.3 min; and (S)-(−)-form: 23.7 min

### Compound 2

### (S)-(−)-trans-{4-[({2-[({1-[3,5-bis(trifluoromethyl)phenyl]ethyl}{5-[2-(methylsulfonyl)ethoxy]pyrimidin-2-yl}amino)methyl]-4-(trifluoromethyl)phenyl}(ethyl)amino)methyl]cyclohexyl}ac etic acid

¹H-NMR (CDCl₃) δ: 0.80-0.96 (7H, m), 1.38 (1H, m), 1.47 (3H, d, J=7.1 Hz), 1.65-1.77 (5H, m), 2.19 (2H, d, J=6.8 Hz), 2.72 (1H, m), 2.81-2.91 (3H, m), 3.08 (3H, s), 3.45 (2H, t, J=5.4 Hz), 4.44 (2H, t, J=5.4 Hz), 4.62 (1H, d, J=17.1 Hz), 4.86 (1H, d, J=17.1 Hz), 6.21 (1H, q, J=7.1 Hz), 7.13 (1H, d, J=8.3 Hz), 7.19 (1H, s), 7.38 (1H, d, J=8.3 Hz), 7.71 (1H, s), 7.73 (2H, s), 8.15 (2H, s).

[α]_{D}²⁰=−46.68 (c=1.0, CHCl₃)

### Compound 3

### (R)-(+)-trans-{4-[({2-[({1-[3,5-bis(trifluoromethyl)phenyl]ethyl}{5-[2-(methylsulfonyl)ethoxy]pyrimidin-2-yl}amino)methyl]-4-(trifluoromethyl)phenyl}(ethyl)amino)methyl]cyclohexyl}ac etic acid

¹H-NMR (CDCl₃) S: the same as Compound 2

[_{α}]D²⁰=+48.92 (c=1.0, CHCl₃)

### Production Example 2

### Production of Substantially Optically Pure (S)-Form Compound 2 According to Preferential Crystallization

An outline of a method for producing a substantially optically pure (S)-form Compound 2 according to preferential crystallization, which was carried out by the present inventors, will be shown as Scheme 1 below.

The absolute configuration of each compound was determined on the basis of the absolute configuration of (R)-1-bromo-1-[3,5-bis(trifluoromethyl)phenyl]ethane confirmed by Step 1.

Moreover, the optical purity of the (S)-form Compound 2 obtained in Step 6, (S)-trans-{4-[({2-[({1-[3,5-bis(trifluoromethyl)phenyl]ethyl}{5-[2-(methylsulfonyl)ethoxy]pyrimidin-2-yl}amino)methyl]-4-(trifluoromethyl)phenyl}(ethyl)amino)methyl]cyclohexyl}ac etic acid, was determined by chiral HPLC analysis under the conditions described in the above described Production Example 1.

Furthermore, the optical purity of each of 1-bromo-1-[3,5-bis(trifluoromethyl)phenyl]ethane obtained in Step 1 and trans-{4-[({2-[({1-[3,5-bis(trifluoromethyl)phenyl]ethyl}{5-[2-(methylsulfonyl)ethoxy]pyrimidin-2-yl}amino)methyl]-4-(trifluoromethyl)phenyl}(ethyl)amino)methyl]cyclohexyl}ac etic acid benzyl ester obtained in Steps 4 and 5 was determined by chiral HPLC analysis under the following conditions.

Conditions for chiral HPLC analysis of 1-bromo-1-[3,5-bis(trifluoromethyl)phenyl]ethane

### Column: CHIRALPAK AS-RH

Mobile phase: ethanol/water=60/40
Flow rate: 0.5 mL/min
Column temperature: 25° C.
Detection wavelength: 220 nm
Retention time: first peak/21.8 min ((R)-form), second peak/26.0 min ((S)-form)
Conditions for chiral HPLC analysis of trans-{4-[({2-[({1-[3,5-bis(trifluoromethyl)phenyl]ethyl}{5-[2-(methylsulfonyl)ethoxy]pyrimidin-2-yl}amino)methyl]-4-(trifluoromethyl)phenyl}(ethyl)amino)methyl]cyclohexyl}ac etic acid benzyl ester

### Column: CHIRALCEL OD-H

Mobile phase: hexane/ethanol=80/20
Flow rate: 1.0 mL/min
Column temperature: 40° C.
Detection wavelength: 242 nm
Retention time: first peak/11.3 min ((R)-form), second peak/13.0 min ((S)-form)

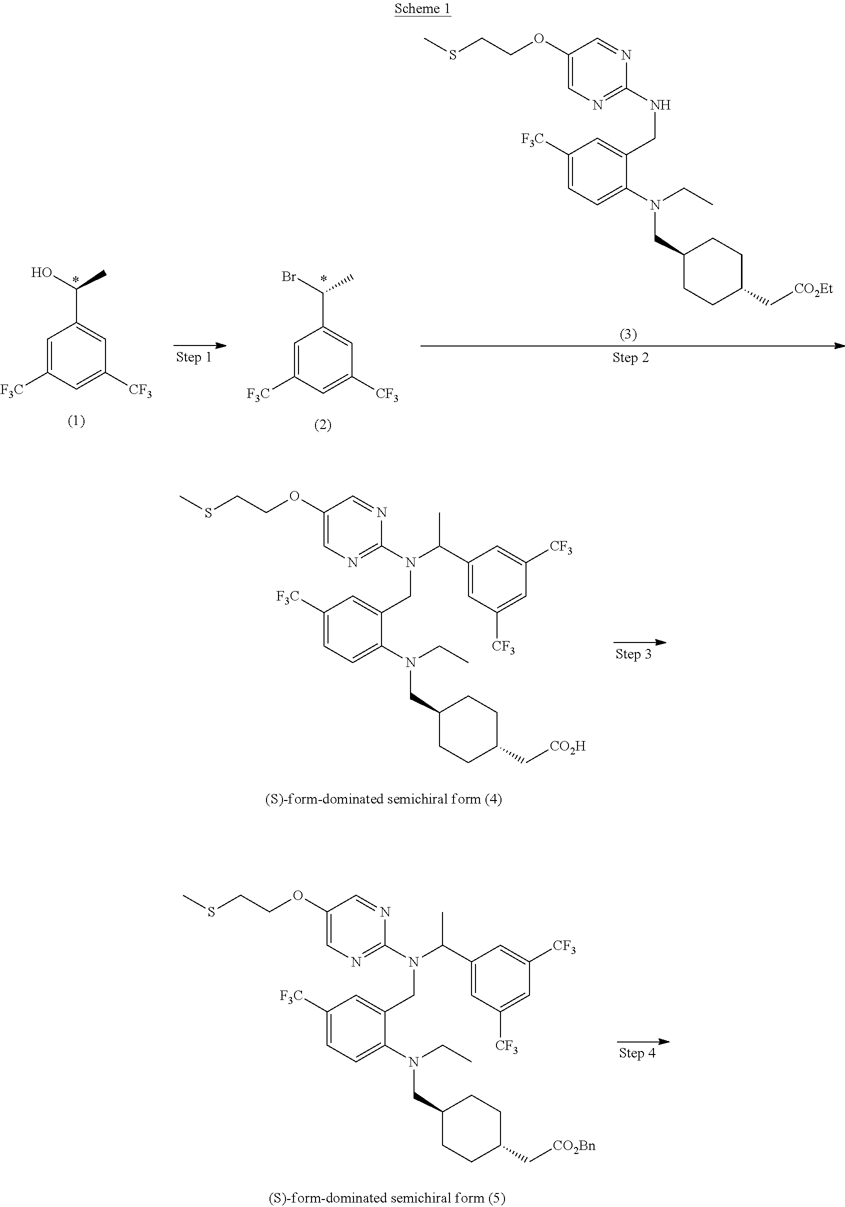 [SMILES: C[C@H](O)c1cc(C(F)(F)F)cc(C(F)(F)F)c1 ; C[C@@H](Br)c1cc(C(F)(F)F)cc(C(F)(F)F)c1 ; CCOC(=O)C[C@H]1CC[C@H](CN(CC)c2ccc(C(F)(F)F)cc2CNc2ncc(OCCSC)cn2)CC1 ; CCN(C[C@H]1CC[C@H](CC(=O)O)CC1)c1ccc(C(F)(F)F)cc1CN(c1ncc(OCCSC)cn1)C(C)c1cc(C(F)(F)F)cc(C(F)(F)F)c1 ; CCN(C[C@H]1CC[C@H](CC(=O)OCc2ccccc2)CC1)c1ccc(C(F)(F)F)cc1CN(c1ncc(OCCSC)cn1)C(C)c1cc(C(F)(F)F)cc(C(F)(F)F)c1]

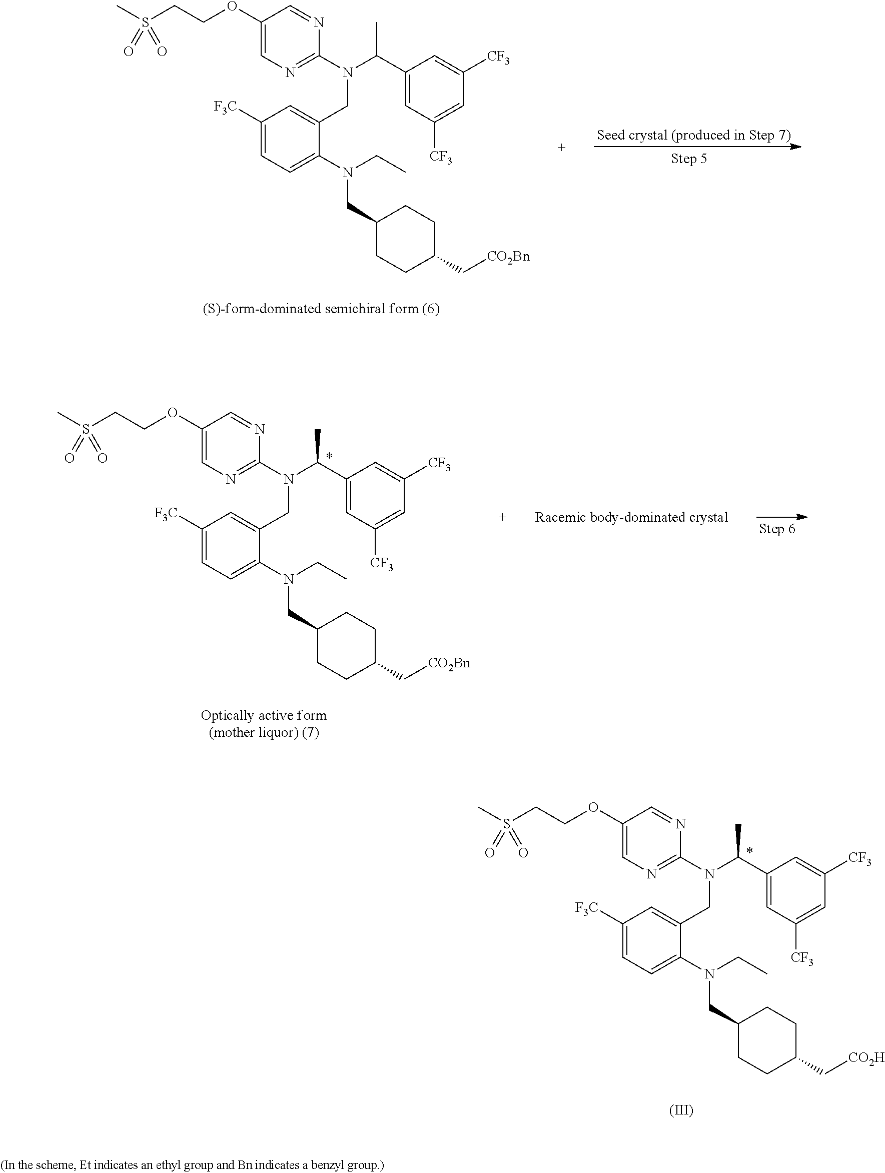 [SMILES: CCN(C[C@H]1CC[C@H](CC(=O)OCc2ccccc2)CC1)c1ccc(C(F)(F)F)cc1CN(c1ncc(OCCS(C)(=O)=O)cn1)C(C)c1cc(C(F)(F)F)cc(C(F)(F)F)c1 ; CCN(C[C@H]1CC[C@H](CC(=O)OCc2ccccc2)CC1)c1ccc(C(F)(F)F)cc1CN(c1ncc(OCCS(C)(=O)=O)cn1)[C@@H](C)c1cc(C(F)(F)F)cc(C(F)(F)F)c1 ; CCN(C[C@H]1CC[C@H](CC(=O)O)CC1)c1ccc(C(F)(F)F)cc1CN(c1ncc(OCCS(C)(=O)=O)cn1)[C@@H](C)c1cc(C(F)(F)F)cc(C(F)(F)F)c1 ; "(III)"]

### Step 1: Production of (R)-1-bromo-1-[3,5-bis(trifluoromethyl)phenyl]ethane

(R)-1-bromo-1-[3,5-bis(trifluoromethyl)phenyl]ethane was produced by the method described in 1-(a) below, and the absolute configuration thereof was then confirmed as follows. Specifically, the obtained (R)-1-bromo-1-[3,5-bis(trifluoromethyl)phenyl]ethane was induced to (S)-1-[3,5-bis(trifluoromethyl)phenyl]ethylamine. Thereafter, the absolute configuration of the thus induced compound was confirmed by comparing the compound with a commercially available standard product of (S)-1-[3,5-bis(trifluoromethyl)phenyl]ethylamine whose absolute configuration had been known, in terms of the sign in the measured value of specific optical rotation.

Moreover, (R)-1-bromo-1-[3,5-bis(trifluoromethyl)phenyl]ethane was also produced by the method described in 1-(b) below, separately.

### 1-(a): Production 1 of (R)-1-bromo-1-[3,5-bis(trifluoromethyl)phenyl]ethane

Under an argon atmosphere, 1,2-dibromo-1,1,2,2-tetrachloroethane (7.57 g, 23.2 mmol) was dissolved in toluene (12.5 mL), and triphenylphosphine (6.1 g, 23.2 mmol) was then added to the obtained solution at 0° C., followed by stirring for 30 minutes. Thereafter, a toluene solution (12.5 mL) containing (S)-1-[3,5-bis(trifluoromethyl)phenyl]ethanol (1) (5.0 g, 19.4 mmol, >99.5% ee) was added dropwise to the resulting solution at 0° C. over 10 or more minutes. Thereafter, the temperature of the mixed solution was increased to a room temperature, and the solution was then stirred at the same temperature as described above for 1 hour. Subsequently, n-hexane (25 mL) was added to the reaction solution, followed by filtration with Celite. The filtrate was successively washed with water, saturated sodium bicarbonate water, and saturated saline solution, and it was then dried over sodium sulfate. Thereafter, it was distilled away under a reduced pressure. The obtained residue was subjected to distillation under a reduced pressure (56° C., 0.7 mmHg) to obtain 5.52 g of (R)-1-bromo-1-[3,5-bis(trifluoromethyl)phenyl]ethane (2) in the form of a colorless oily product (yield: 88.6%).

Chiral HPLC analysis: optical purity >99.5% ee (main peak: first peak), invert ratio ≧99%
[α]_{D}²⁵+59.1 (c=1.03, CHCl₃)

¹H-NMR (CDCl₃) δ: 2.08 (3H, d, J=7.1 Hz), 5.21 (1H, q, J=7.1 Hz), 7.81 (1H, s), 7.87 (2H, s)

### Confirmation of absolute configuration of (R)-1-bromo-1-[3,5-bis(trifluoromethyl)phenyl]ethane

Sodium azide (64.4 mg, 0.990 mmol) was added to an N,N-dimethylformamide solution (1 mL) containing (R)-1-bromo-1-[3,5-bis(trifluoromethyl)phenyl]ethane (2) (106 mg, 0.336 mmol, 99% ee) obtained in 1-(a) above, and the obtained mixture was then stirred at −18° C. to −15° C. for 4 hours. Thereafter, the reaction solution was extracted with ethyl acetate/n-hexane (1:1) and water. The organic layer was washed with saturated saline solution, and was then dried over anhydrous sodium sulfate, followed by concentration under a reduced pressure, to obtain 111.5 mg of 1-[3,5-bis(trifluoromethyl)phenyl]ethyl azide (crude product: 111.5 mg).

¹H-NMR (CDCl₃) δ: 1.61 (3H, d, J=6.8 Hz), 4.79 (1H, q, J=6.8 Hz), 7.78 (2H, s), 7.84 (1H, s)

The obtained 1-[3,5-bis(trifluoromethyl)phenyl]ethyl azide (crude product: 111.5 mg) was dissolved in methanol (6 mL), and palladium fibroin (18 mg) was then added to the obtained solution. Hydrogen substitution was carried out and the mixture was stirred at a room temperature for 1 hour. The reaction solution was filtrated with Celite, and the filtrate was then concentrated under a reduced pressure. The obtained residue was purified by silica gel column chromatography (chloroform:methanol=50:1 to 5:1), to obtain 77.6 mg of 1-[3,5-bis(trifluoromethyl)phenyl]ethylamine in the form of a colorless oily product (yield: 91%, 2 steps).

¹H-NMR (CDCl₃) δ: 1.42 (3H, d, J=6.8 Hz), 1.58 (2H, br-s), 4.30 (1H, q, J=6.8 Hz), 7.75 (1H, s), 7.85 (2H, s)

The specific optical rotation of the obtained 1-[3,5-bis(trifluoromethyl)phenyl]ethylamine is the following.

[α]_{D}²⁵−15.9 (c=1.31, CHCl₃)

On the other hand, the specific rotation of a commercially available standard product of (S)-1-[3,5-bis(trifluoromethyl)phenyl]ethylamine (manufactured by Central Glass Co., Ltd.; Lot. 0102000; optical purity: 99% ee) is the following.

[α]_{D}²⁵−15.9 (c=1.15, CHCl₃)

The sign in the measured value of the specific optical rotation was identical with that of the commercially available standard product. Thus, the obtained 1-[3,5-bis(trifluoromethyl)phenyl]ethylamine was confirmed to be an (S)-form. Since this amine was obtained from 1-bromo-1-[3,5-bis(trifluoromethyl)phenyl]ethane via the nucleophilic substitution reaction of azide ions, the 1-bromo-1-[3,5-bis(trifluoromethyl)phenyl]ethane obtained in 1-(a) above was confirmed to be an (R)-form.

### 1-(b): Production 2 of (R)-1-bromo-1-[3,5-bis(trifluoromethyl)phenyl]ethane

Under an argon atmosphere, phosphorus tribromide (157.3 g, 0.58 mol) was added dropwise to (S)-1-[3,5-bis(trifluoromethyl)phenyl]ethanol (1) (300 g, 1.16 mol, 96% ee) at 20° C. or lower in a water bath, and the obtained mixture was then stirred at 19° C. to 22° C. for 30 minutes. Thereafter, the reaction solution was cooled, and hydrogen bromide (30% acetic acid solution) (228 mL, 1.16 mol) was added dropwise thereto at 0° C. or lower. The obtained mixture was stirred at 13° C. to 15° C. for 16 hours. Thereafter, the reaction solution was added to ice water, and was then extracted with n-hexane (3 L×2). The organic layers were gathered. The gathered organic layer was successively washed with saturated sodium bicarbonate water (3 L) and saturated saline solution (3 L), and was then dried over anhydrous magnesium sulfate. The resultant was concentrated under a reduced pressure (90 to 100 mmHg) to obtain 389.2 g of a crude product. The obtained crude product was purified by column chromatography (900 g of silica gel, developing solvent: n-hexane), to obtain 349.8 g of (R)-1-bromo-1-[3,5-bis(trifluoromethyl)phenyl]ethane (2) in the form of a colorless oily product (yield: 93.8%).

Since the first peak appeared as a main peak in the chiral HPLC analysis, as shown below, 1-bromo-1-[3,5-bis(trifluoromethyl)phenyl]ethane produced in 1-(b) above was also confirmed to be an (R)-form, as with the compound obtained in 1-(a) above.

Chiral HPLC analysis: optical purity >93.9% ee (main peak: first peak); invert ratio: 97.8%

¹H-NMR (CDCl₃) δ: 2.08 (3H, d, J=7.1 Hz), 5.21 (1H, q, J=7.1 Hz), 7.81 (1H, s), 7.87 (2H, s)

### Step 2: Production of (S)-form-dominated semichiral form of trans-{4-[({2-[({1-[3,5-bis(trifluoromethyl)phenyl]ethyl}{5-[2-(methylthio)ethoxy]pyrimidin-2-yl}amino)methyl]-4-(trifluoromethyl)phenyl}(ethyl)amino)methyl]cyclohexyl}ac etic acid

Under an argon atmosphere, NaH (60% in oil, 119 g, 2.98 mol) was added to an anhydrous tetrahydrofuran (THF, 2.26 L) solution containing trans-[4-([(ethyl){2-[({5-[2-(methylthio)ethoxy]pyrimidin-2-yl}amino)methyl]-4-(trifluoromethyl)phenyl}amino]methyl)cyclohexyl]ethyl acetate (3) (565.4 g, 0.99 mol) synthesized by the method described in Patent Literature 2 (International Publication No. WO2008/129951) under cooling on ice, and the obtained mixture was then stirred at a room temperature for 1 hour. Thereafter, the reaction solution was cooled to −30° C., and an anhydrous N,N-dimethylformamide (4.53 L) solution of (R)-1-bromo-1-[3,5-bis(trifluoromethyl)phenyl]ethane (2) (682 g, 1.99 mol, 93.9% ee) obtained in Step 1 was then added dropwise to the reaction solution, so that the temperature in the reaction system became −15° C. or lower. The obtained mixture was stirred at −15° C. to −1° C. for 5 hours. Thereafter, the reaction solution was added to a mixed solution of ice water (35 L) and toluene (30 L). After that, citric acid was added to the mixed solution until the pH was adjusted to 6.9, and an organic layer was separated.

A water layer was extracted with toluene (20 L) twice, and the organic layers were then gathered. The gathered organic layer was dried over anhydrous magnesium sulfate, and was then concentrated under a reduced pressure to obtain a crude product. The crude product was dissolved in ethanol (8 L), and a 2 M NaOH aqueous solution (1.24 L, 2.48 mol) was added to the obtained solution under cooling on ice. The obtained mixture was stirred at 50° C. for 3.5 hours. Under cooling on ice, a 1 M HCl aqueous solution was added to the reaction solution until the pH was adjusted to 5.4. The thus mixed solution was poured into water (25 L), and extraction was then carried out with ethyl acetate (22 L) twice. The organic layer was washed with saturated saline solution (12 L), and was then dried over anhydrous magnesium sulfate, followed by concentration under a reduced pressure. The obtained residue was purified by column chromatography (21 g of silica gel, developing solvent: heptane/acetone=7/1→3/1), to obtain a semichiral form (4) of trans-{4-[({2-[({1-[3,5-bis(trifluoromethyl)phenyl]ethyl}{5-[2-(methylthio)ethoxy]pyrimidin-2-yl}amino)methyl]-4-(trifluoromethyl)phenyl}(ethyl)amino)methyl]cyclohexyl}ac etic acid (yellow oily product, 744.1 g, yield: 96%).

As described in Step 1 above, (R)-1-bromo-1-[3,5-bis(trifluoromethyl)phenyl]ethane (2), the absolute configuration of which had been confirmed, was used as a raw material, and a nucleophilic substitution reaction with amine (3) progressed. Thus, the (S)-form is dominated in the obtained semichiral form (4).

¹H-NMR (CDCl₃) δ: 0.85-0.96 (7H, m), 1.35-1.45 (4H, m), 1.60-1.78 (5H, m), 2.18-2.21 (5H, m), 2.69 (1H, m), 2.81-2.91 (5H, m), 4.16 (2H, q, J=6.8 Hz), 4.61 (1H, d, J=17.1 Hz), 4.85 (1H, d, J=17.1 Hz), 6.22 (1H, q, J=6.8 Hz), 7.11 (1H, d, J=8.6 Hz), 7.23 (1H, s), 7.37 (1H, d, J=8.3 Hz), 7.70 (1H, s), 7.73 (2H, s), 8.14 (2H, s)

### Step 3: Production of (S)-form-dominated semichiral form of trans-{4-[({2-[({1-[3,5-bis(trifluoromethyl)phenyl]ethyl}{5-[2-(methylthio)ethoxy]pyrimidin-2-yl}amino)methyl]-4-(trifluoromethyl)phenyl}(ethyl)amino)methyl]cyclohexyl}ac etic acid benzyl ester

Under an argon atmosphere, benzyl alcohol (113.1 g, 1.05 mol), WS.HCl (200.5 g, 1.05 mol) and DMAP (11.9 g, 98 mmol) were added to an anhydrous dichloroethane (11.6 L) solution containing the (S)-form-dominated semichiral form (4) (744.1 g, 0.95 mol) of trans-{4-[({2-[({1-[3,5-bis(trifluoromethyl)phenyl]ethyl}{5-[2-(methylthio)ethoxy]pyrimidin-2-yl}amino)methyl]-4-(trifluoromethyl)phenyl}(ethyl)amino)methyl]cyclohexyl}ac etic acid obtained in Step 2 under cooling on ice. The thus obtained mixture was stirred at a room temperature overnight. Thereafter, water (10 L) was added to the reaction solution, and extraction was then carried out with chloroform (19 L, 14 L). The organic layer was washed with saturated saline solution (12 L), and was then dried over anhydrous magnesium sulfate, followed by concentration under a reduced pressure. The obtained residue was purified by column chromatography (28 g of silica gel, developing solvent: heptane/ethyl acetate=6/1), to obtain a semichiral form (5) of trans-{4-[({2-[({1-[3,5-bis(trifluoromethyl)phenyl]ethyl}{5-[2-(methylthio)ethoxy]pyrimidin-2-yl}amino)methyl]-4-(trifluoromethyl)phenyl}(ethyl)amino)methyl]cyclohexyl}ac etic acid benzyl ester (yellow oily product, 745.8 g, yield: 90%).

It is to be noted that (S)-form is dominated in the obtained semichiral form (5), as with the semichiral form (4).

¹H-NMR (CDCl₃) δ: 0.87-0.95 (7H, m), 1.37 (1H, m), 1.43 (3H, d, J=7.1 Hz), 1.65-1.77 (5H, m), 2.20 (2H, d, J=6.8 Hz), 2.22 (3H, s), 2.66-2.71 (2H, m), 2.82-2.91 (4H, m), 4.15 (2H, t, J=6.6 Hz), 4.62 (1H, d, J=17.1 Hz), 4.85 (1H, d, J=17.1 Hz), 5.10 (2H, s), 6.21 (1H, q, J=7.1 Hz), 7.10 (1H, d, J=8.3 Hz), 7.22 (1H, s), 7.28-7.38 (6H, m), 7.70 (1H, s), 7.73 (2H, s), 8.14 (2H, s)

### Step 4: Production of (S)-form-dominated semichiral form of trans-{4-[({2-[({1-[3,5-bis(trifluoromethyl)phenyl]ethyl}{5-[2-(methylsulfonyl)ethoxy]pyrimidin-2-yl}amino)methyl]-4-(trifluoromethyl)phenyl}(ethyl)amino)methyl]cyclohexyl}ac etic acid benzyl ester

Under an argon atmosphere, tantalum pentachloride (31.3 g, 87.3 mmol) and 30% hydrogen peroxide water (496 mL, 4.38 mol) were added to a 2-propanol (15.2 L) solution containing (S)-form-dominated semichiral form (5) (745.8 g, 0.87 mol) of the trans-{4-[({2-[({1-[3,5-bis(trifluoromethyl)phenyl]ethyl}{5-[2-(methylthio)ethoxy]pyrimidin-2-yl}amino)methyl]-4-(trifluoromethyl)phenyl}(ethyl)amino)methyl]cyclohexyl}ac etic acid benzyl ester obtained in Step 3. The thus obtained mixture was stirred at a room temperature for 5 hours. Thereafter, the reaction solution was quenched with a saturated sodium hydrogen sulfite aqueous solution (3.1 L), and water (15 L) was then added to the reaction solution. The mixed solution was extracted with chloroform (14 L, 12 L). The organic layer was washed with saturated saline solution (20 L), and was then dried over anhydrous magnesium sulfate, followed by concentration under a reduced pressure. The obtained residue was purified by column chromatography (26 kg of silica gel, developing solvent: heptane/ethyl acetate=3/1→2/1), so as to obtain a semichiral form (6) of the trans-{4-[({2-[({1-[3,5-bis(trifluoromethyl)phenyl]ethyl}{5-[2-(methylsulfonyl)ethoxy]pyrimidin-2-yl}amino)methyl]-4-(trifluoromethyl)phenyl}(ethyl)amino)methyl]cyclohexyl}ac etic acid benzyl ester (yellow amorphous product, 619.5 g, yield: 79%).

It is to be noted that (S)-form is dominated in the obtained semichiral form (6), as with the semichiral form (4) and the semichiral form (5).

Chiral HPLC analysis: optical purity 67.7% ee (main peak: second peak)

¹H-NMR (CDCl₃) δ: 0.87-0.96 (7H, m), 1.38 (1H, m), 1.45 (3H, d, J=7.1 Hz), 1.65-1.80 (5H, m), 2.21 (2H, d, J=6.6 Hz), 2.69 (1H, m), 2.81-2.91 (3H, m), 3.08 (3H, s), 3.44 (2H, t, J=5.4 Hz), 4.44 (2H, t, J=5.4 Hz), 4.64 (1H, d, J=17.1 Hz), 4.86 (1H, d, J=17.3 Hz), 5.10 (2H, s), 6.19 (1H, q, J=6.9 Hz), 7.12 (1H, d, J=8.3 Hz), 7.19 (1H, s), 7.30-7.39 (6H, m), 7.71 (1H, s), 7.72 (2H, s), 8.16 (2H, s)

### Step 5: Production of substantially optically pure (S)-trans-{4-[({2-[({1-[3,5-bis(trifluoromethyl)phenyl]ethyl}{5-[2-(methylsulfonyl)ethoxy]pyrimidin-2-yl}amino)methyl]-4-(trifluoromethyl)phenyl}(ethyl)amino)methyl]cyclohexyl}ac etic acid benzyl ester

The (S)-form-dominated semichiral form (6) (111.7 g, 123.7 mmol, 67.7% ee) of trans-{4-[({2-[({1-[3,5-bis(trifluoromethyl)phenyl]ethyl}{5-[2-(methylsulfonyl)ethoxy]pyrimidin-2-yl}amino)methyl]-4-(trifluoromethyl)phenyl}(ethyl)amino)methyl]cyclohexyl}ac etic acid benzyl ester obtained in Step 4 was dissolved in ethanol (825 mL). Then, 2.0 mg of a seed crystal (a racemic crystal produced in Step 7 below), which had been prepared separately, was added to the above obtained solution at 15° C. to 20° C. The obtained mixture was stirred at the same temperature as described above for 21 hours, and then at 0° C. for 3 hours. Thereafter, the precipitate was separated by filtration, and was then washed with cold ethanol (165 mL), followed by concentration of mother liquor under a reduced pressure, to obtain substantially optically pure trans-{4-[({2-[({1-[3,5-bis(trifluoromethyl)phenyl]ethyl}{5-[2-(methylsulfonyl)ethoxy]pyrimidin-2-yl}amino)methyl]-4-(trifluoromethyl)phenyl}(ethyl)amino)methyl]cyclohexyl}ac etic acid benzyl ester (7) (yellow amorphous product, 66.38 g, yield: 59%).

It is to be noted that the obtained trans-{4-[({2-[({1-[3,5-bis(trifluoromethyl)phenyl]ethyl}{5-[2-(methylsulfonyl)ethoxy]pyrimidin-2-yl}amino)methyl]-4-(trifluoromethyl)phenyl}(ethyl)amino)methyl]cyclohexyl}ac etic acid benzyl ester (7) is an (S)-form because it was obtained by separating a racemic body-dominated crystal from the (S)-form-dominated semichiral form (6) by filtration.

Chiral HPLC analysis: optical purity >99% ee (main peak: second peak)
[α]_{D}²⁰−42.36 (c=1.0 w/v %, CHCl₃)

As a result of the chiral HPLC analysis, the optical purity of the filtrated racemic body-dominated crystal was found to be 22% ee (43.39 g, yield: 39%).

### Step 6: Production of substantially optically pure (S)-trans-{4-[({2-[({1-[3,5-bis(trifluoromethyl)phenyl]ethyl}{5-[2-(methylsulfonyl)ethoxy]pyrimidin-2-yl}amino)methyl]-4-(trifluoromethyl)phenyl}(ethyl)amino)methyl]cyclohexyl}ac etic acid

Under a nitrogen atmosphere, 10% Pd—C (wet) (3.4 g) was added to an ethanol (340 mL) solution containing (S)-trans-{4-[({2-[({1-[3,5-bis(trifluoromethyl)phenyl]ethyl}{5-[2-(methylsulfonyl)ethoxy]pyrimidin-2-yl}amino)methyl]-4-(trifluoromethyl)phenyl}(ethyl)amino)methyl]cyclohexyl}ac etic acid benzyl ester (7) (34.2 g, 37.88 mmol, >99% ee) obtained in Step 5. After hydrogen substitution was carried out, the reaction mixture was stirred at a room temperature for 2 hours. Thereafter, the reaction suspension was filtrated with Celite, and was then washed with ethanol (50 mL). After that, the wash liquid was concentrated under a reduced pressure to obtain substantially optically pure trans-{4-[({2-[({1-[3,5-bis(trifluoromethyl)phenyl]ethyl}{5-[2-(methylsulfonyl)ethoxy]pyrimidin-2-yl}amino)methyl]-4-(trifluoromethyl)phenyl}(ethyl)amino)methyl]cyclohexyl}ac etic acid (Compound 2) (white amorphous product, 31.78 g, yield: 100%).

As shown in the following specific optical rotation, the obtained compound was a levorotatory compound. In addition, since the compound was obtained by deprotecting ester from the benzyl ester (7) of an (S)-form, it is also an (S)-form.

Chiral HPLC analysis: optical purity >99% ee (main peak: second peak)
[α]_{D}²⁰−46.68 (c=1.0, CHCl₃)
IR (ATR) cm⁻¹: 2921, 1706, 1479, 1279, 1134

¹H-NMR (CDCl₃) δ: 0.80-0.96 (7H, m), 1.38 (1H, m), 1.47 (3H, d, J=7.1 Hz), 1.65-1.77 (5H, m), 2.19 (2H, d, J=6.8 Hz), 2.72 (1H, m), 2.81-2.91 (3H, m), 3.08 (3H, s), 3.45 (2H, t, J=5.2 Hz), 4.44 (2H, q, J=5.4 Hz), 4.62 (1H, d, J=17.1 Hz), 4.86 (1H, d, J=17.4 Hz), 6.21 (1H, q, J=7.1 Hz), 7.13 (1H, d, J=8.3 Hz), 7.19 (1H, s), 7.38 (1H, d, J=6.6 Hz), 7.71 (1H, s), 7.73 (2H, s), 8.15 (2H, s)

### Step 7: Production of racemic seed crystal of trans-{4-[({2-[({1-[3,5-bis(trifluoromethyl)phenyl]ethyl}{5-[2-(methylsulfonyl)ethoxy]pyrimidin-2-yl}amino)methyl]-4-(trifluoromethyl)phenyl}(ethyl)amino)methyl]cyclohexyl}ac etic acid benzyl ester

Under cooling on ice, benzyl alcohol (2.93 g, 27.07 mmol), DMAP (300 mg, 2.46 mmol) and WS.HCl (5.19 g, 27.07 mmol) were added to an anhydrous dichloromethane (200 mL) solution containing trans-{4-[({2-[({1-[3,5-bis(trifluoromethyl)phenyl]ethyl}{5-[2-(methylsulfonyl)ethoxy]pyrimidin-2-yl}amino)methyl]-4-(trifluoromethyl)phenyl}(ethyl)amino)methyl]cyclohexyl}ac etic acid (racemic compound (I)) (20 g, 24.61 mmol) synthesized by the method described in Example 45 of Patent Literature 2 (International Publication No. WO2008/129951). The temperature of the thus obtained mixture was increased to a room temperature, and the mixture was then stirred for 16 hours. Thereafter, water (100 mL) was added to the reaction solution, and extraction was then carried out with chloroform (500 mL). The organic layer was washed with a 2 M hydrochloric acid aqueous solution (100 mL) and saturated saline solution (100 mL), and was then dried over anhydrous magnesium sulfate, followed by concentration under a reduced pressure. The obtained residue was purified by column chromatography (350 g of silica gel, developing solvent: N-hexane/ethyl acetate=3/1→1/1), to obtain trans-{4-[({2-[({1-[3,5-bis(trifluoromethyl)phenyl]ethyl}{5-[2-(methylsulfonyl)ethoxy]pyrimidin-2-yl}amino)methyl]-4-(trifluoromethyl)phenyl}(ethyl)amino)methyl]cyclohexyl}ac etic acid benzyl ester (21.15 g, yield: 95.2%) in the form of a white amorphous product.

The obtained white amorphous trans-{4-[({2-[({1-[3,5-bis(trifluoromethyl)phenyl]ethyl}{5-[2-(methylthio)ethoxy]pyrimidin-2-yl}amino)methyl]-4-(trifluoromethyl)phenyl}(ethyl)amino)methyl]cyclohexyl}ac etic acid benzyl ester (7.9 g) was dissolved in ethanol (40 mL), and the obtained solution was then stirred at a room temperature for 15 hours. Thereafter, the obtained precipitate was collected by filtration, and it was washed with cold ethanol (20 mL) and was then dried at 60° C. for 4 hours under a reduced pressure, so as to obtain a racemic crystal of trans-{4-[({2-[({1-[3,5-bis(trifluoromethyl)phenyl]ethyl}{5-[2-(methylsulfonyl)ethoxy]pyrimidin-2-yl}amino)methyl]-4-(trifluoromethyl)phenyl}{ethyl)amino)methyl]cyclohexyl}ac etic acid benzyl ester (white crystalline powder, 6.98 g, recovery rate: 88.4%).

In the present Production Example 2, a compound having a middle level of optical purity obtained as a result of a reduction in the optical purity caused by partial racemization (a compound having an optical purity of approximately 50 to 90% ee, and preferably approximately 70 to 90% ee) is referred to as a “semichiral form.” Furthermore, the semichiral form, in which a compound with S-configured asymmetric carbon atom(s) is present in an amount excessively larger than a compound with R-configured asymmetric carbon atom(s), is referred to as an “(S)-form-dominated semichiral form.”

### Test Example 1

A compound in a test compound group was added to cells of a human hepatocellular carcinoma cell line HepG2, and the cells were then cultured for 24 hours. Thereafter, the expression levels of NPC1L1 and LIPG mRNAs were measured by real-time RT-PCR. Specifically, the HepG2 cells were inoculated at 2×10⁵ cells/well into a 24-well plate, and were then cultured overnight. Thereafter, Compound 2 was dissolved in dimethyl sulfoxide (DMSO) to give concentrations of 0.1 μM, 1 μM and 10 μM, and Anacetrapib was dissolved in DMSO to give concentrations of 1 μM and 10 μM. The thus prepared solution was added to the culture in an amount of 1/1000-fold of the culture fluid. The cells were cultured at 37° C. for 24 hours in a CO₂ incubator. Thereafter, 500 μL of ISOGEN (Nippon Gene Co., Ltd., Catalog No. 31-02501) was added to the culture, and total RNA was then extracted. From the extracted total RNA, cDNA was synthesized using High Capacity cDNA Reverse Transcription kit (Applied Biosystems, Catalog No. 4368813). The expression levels of human NPC1L1 and LIPG mRNAs were measured, using human NPC1L1-specific primers (sense: 5′-CAGGTATGGTCGCCCGAAGCAC-3′ (SEQ ID NO: 1); and antisense: 5′-TGCGGTTGTTCTGGAAATACTG-3′ (SEQ ID NO: 2)), human LIPG-specific primers (sense: 5′-TCAACGATGTCTTGGGATCA-3′ (SEQ ID NO: 3); and antisense: 5′-TGAAGCGATTGGAGTCAGTG-3′ (SEQ ID NO: 4)), and Fast SYBR Green master mix (Applied Biosystems, Catalog No. 4385614). 7900HT Fast Realtime PCR system was used as a measurement device.

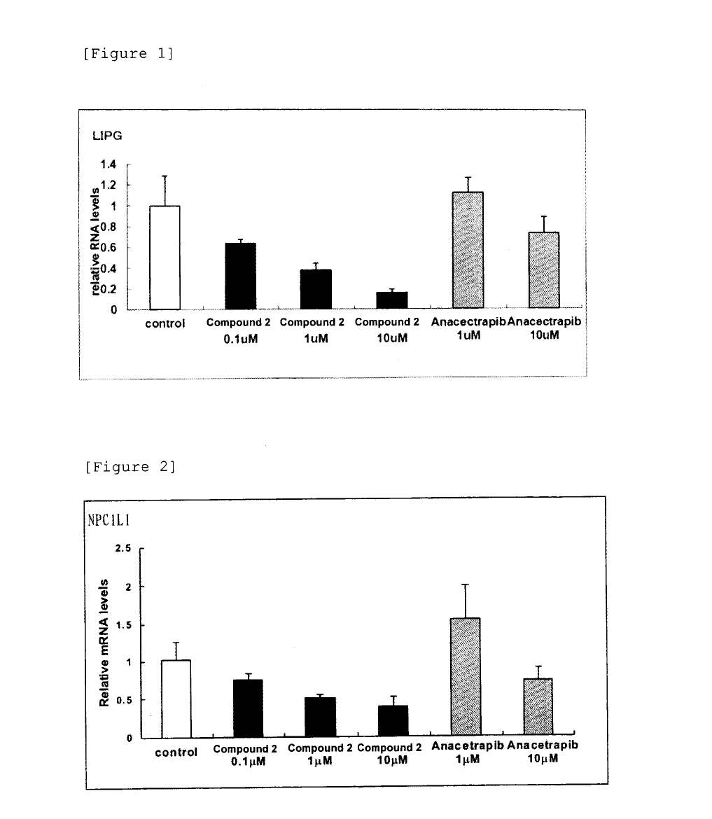

The measurement value was corrected with the expression level of β-Actin mRNA. The expression levels of LIPG and NPC1L1 mRNAs in cells, to which only DMSO had been added, were defined as 1, and the expression levels of LIPG and NPC1L1 mRNAs in cells, to which the test compound group (Compound 2 or Anacetrapib) had been added, were calculated as a relative value thereof. The results are shown in FIGS. 1 and 2.

### Test Example 2

A compound of a test compound group was added to cells of a human hepatocellular carcinoma cell line HepG2, and the cells were then cultured for 24 hours. Thereafter, the gene expression profile was measured using the DNA microarray of Agilent. Specifically, the HepG2 cells were inoculated at a concentration of 2×10⁵ cells/well into a 24-well plate, and were then cultured overnight. Thereafter, Compound 2 was dissolved in dimethyl sulfoxide (DMSO) to give concentrations of 1 μM and 10 μM, whereas Anacetrapib was dissolved in DMSO to give a concentration of 10 W. The thus prepared solution was added to the culture fluid in an amount of 1/1000-fold of the culture fluid. The cells were cultured at 37° C. for 24 hours in a CO₂ incubator. Thereafter, 500 μL of ISOGEN (Nippon Gene Co., Ltd., Catalog No. 31-02501) was added to the culture, and total RNA was then extracted. Cy3-labeled cRNA was synthesized from 200 ng of the RNA using Low Input Quick Amp Labeling Kit (Agilent, Catalog No. 5190-2308). The synthesized Cy3-labeled cRNA was hybridized to Whole Human Genome (4×44K) (Agilent, Catalog No. G4110F), using GE Hybridization Buffer HI-RPM (Agilent, Catalog No. 5190-0403). After completion of the hybridization, the DNA microarray was washed with Gene Expression Wash Buffer (Agilent, Catalog No. 5188-5327), and data were then scanned with a scanner (Agilent).

The analysis was carried out using GeneSpring (Agilent), and the change rate of the expression level of each gene was calculated.

(Change rate)=(Expression level of each gene in cells treated with compound)+(Expression level of each gene in cells untreated with compound)

The results are shown in Table 1.

TABLE 1

| | Change rate (fold) | | |
|---|---|---|---|
| | Compound 2 | Compound 2 | Anacetrapib |
| | (1 μM) | (10 μM) | (10 μM) |
| NPC1L1 | −2.3 | −3.1 | −1.5 |
| LIPG | −2.5 | −6.2 | −1.9 |

From the above described pharmacological test results, it became clear that the compound represented by the general formula (I) or (II) has a strong and sustained action to suppress the expression of NPC1L1 and LIPG mRNAs.

### INDUSTRIAL APPLICABILITY

A pharmaceutical comprising a compound having CETP inhibitory activity has a strong action to suppress the expression of NPC1L1 and LIPG mRNAs and is useful for preventing and/or treating obesity. Therefore such a pharmaceutical is industrially applicable.

1-30. (canceled)

31. A method for suppressing expression of NPC1L1 and/or LIPG mRNA, comprising:
administering a compound having an activity of inhibiting cholesteryl ester transfer protein, a salt thereof, or a solvate thereof to a subject in need thereof.

32. The method of claim 31, wherein the compound comprises a compound expressed by formula (I) or (II), a salt thereof, or a solvate thereof: 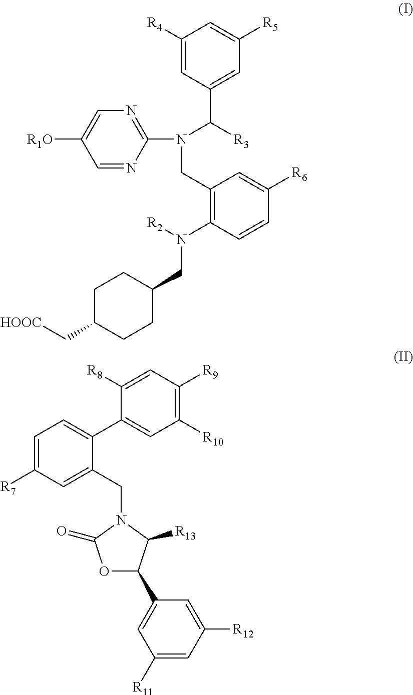
wherein in formula (I);
R₁ is a C₁₋₆ alkylthio C₁₋₆ alkyl group, a C₁₋₆ alkylsulfinyl C₁₋₆ alkyl group or a C₁₋₆ alkylsulfonyl C₁₋₆ alkyl group;
R₂ is a hydrogen atom or an optionally halogen atom-substituted C₁₋₆ alkyl group;
R₃ is an optionally halogen atom-substituted C₁₋₆ alkyl group;
R₄ and R₅, which are the same or different, are each independently a hydrogen atom, a halogen atom, an optionally halogen atom-substituted C₁₋₆ alkyl group, an optionally halogen atom-substituted C₁₋₆ alkoxy group, or a cyano group; and
R₆ is a hydrogen atom, a halogen atom, an optionally halogen atom-substituted C₁₋₆ alkyl group, or an optionally halogen atom-substituted C₁₋₆ alkoxy group; and
in formula (II);
R₇, R₈, R₉, R₁₀, R₁₁, R₁₂ and R₁₃, which are the same or different, are each independently a hydrogen atom, a hydroxy group, a halogen atom, an optionally halogen atom-substituted C₁₋₆ alkyl group, or an optionally halogen atom-substituted C₁₋₆ alkoxy group.

33. The method of claim 31, comprising administering a compound selected from the group consisting of trans-{4-[({2-[({1-[3,5-bis(trifluoromethyl)phenyl]ethyl}{5-[2-(methylsulfonyl)ethoxy]pyrimidin-2-yl}amino)methyl]-4-(trifluoromethyl)phenyl}(ethyl)amino)methyl]cyclohexyl}acetic acid, (S)-(−)-trans-{4-[({2-[({1-[3,5-bis(trifluoromethyl)phenyl]ethyl}{5-[2-(methylsulfonyl)ethoxy]pyrimidin-2-yl}amino)methyl]-4-(trifluoromethyl)phenyl}(ethyl)amino)methyl]cyclohexyl}acetic acid, (R)-(+)-trans-{4-[({2-[({1-[3,5-bis(trifluoromethyl)phenyl]ethyl}{5-[2-(methylsulfonyl)ethoxy]pyrimidin-2-yl}amino)methyl]-4-(trifluoromethyl)phenyl}(ethyl)amino)methyl]cyclohexyl}acetic a salt thereof, a solvate thereof, and a combination thereof.

34. The method of claim 31, wherein the method comprises administering (4S,5R)-5-[3,5-bis(trifluoromethyl)phenyl]-3-((4′-fluoro-5′-isopropyl-2′-methoxy-4-(trifluoromethyl)-[1,1′-biphenyl]-2-yl)methyl)-4-methyloxazolidin-2-one, a salt thereof, or a solvate thereof to a subject in need thereof.

35. A method for suppressing NPC1L1 and/or LIPG protein production, comprising:
administering a compound having an activity of inhibiting cholesteryl ester transfer protein, a salt thereof, or a solvate thereof to a subject in need thereof.

36. The method of claim 35, wherein the compound comprises a compound expressed by formula (I) or (II), a salt thereof, or a solvate thereof: 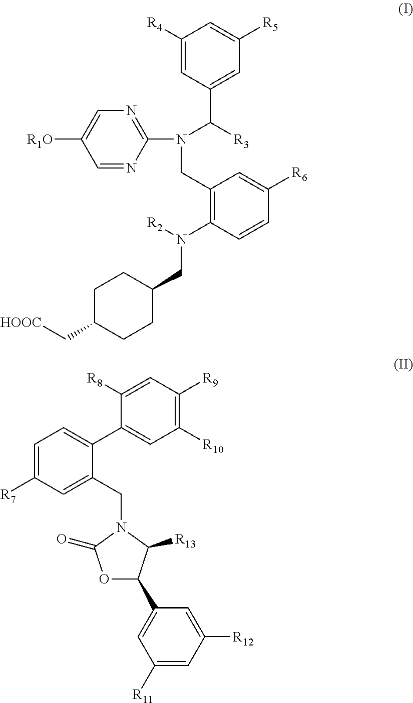
wherein, in formula (I);
R₁ is a C₁₋₆ alkylthio C₁₋₆ alkyl group, a C₁₋₆ alkylsulfinyl C₁₋₆ alkyl group or a C₁₋₆ alkylsulfonyl C₁₋₆ alkyl group;
R₂ is a hydrogen atom or an optionally halogen atom-substituted C₁₋₆ alkyl group;
R₃ is an optionally halogen atom-substituted C₁₋₆ alkyl group;
R₄ and R₅, which are the same or different, are each independently a hydrogen atom, a halogen atom, an optionally halogen atom-substituted C₁₋₆ alkyl group, an optionally halogen atom-substituted C₁₋₆ alkoxy group, or a cyano group; and
R₆ is a hydrogen atom, a halogen atom, an optionally halogen atom-substituted C₁₋₆ alkyl group, or an optionally halogen atom-substituted C₁₋₆ alkoxy group; and
in formula (II);
R₇, R₈, R₉, R₁₀, R₁₁, R₁₂ and R₁₃, which are the same or different, are each independently a hydrogen atom, a hydroxy group, a halogen atom, an optionally halogen atom-substituted C₁₋₆ alkyl group, or an optionally halogen atom-substituted C₁₋₆ alkoxy group.

37. The method of claim 35, wherein the method comprises administering a compound selected from the group consisting of trans-{4-[({2-[({1-[3,5-bis(trifluoromethyl)phenyl]ethyl}{5-[2-(methylsulfonyl)ethoxy]pyrimidin-2-yl}amino)methyl]-4-(trifluoromethyl)phenyl} (ethyl)amino)methyl]cyclohexyl}acetic acid, (S)-(−)-trans-{4-[({2-[({1-[3,5-bis(trifluoromethyl)phenyl]ethyl}{5-[2-(methylsulfonyl)ethoxy]pyrimidin-2-yl}amino)methyl]-4-(trifluoromethyl)phenyl}(ethyl)amino)methyl]cyclohexyl}acetic acid, (R)-(+)-trans-{4-[({2-[({1-[3,5-bis(trifluoromethyl)phenyl]ethyl}{5-[2-(methylsulfonyl)ethoxy]pyrimidin-2-yl}amino)methyl]-4-(trifluoromethyl)phenyl}(ethyl)amino)methyl]cyclohexyl}acetic acid, a salt thereof, a solvate thereof, and a combination thereof.

38. The method of claim 35, wherein the method comprises administering (4S,5R)-5-[3,5-bis(trifluoromethyl)phenyl]-3-((4′-fluoro-5′-isopropyl-2′-methoxy-4-(trifluoromethyl)-[1,1′-biphenyl]-2-yl)methyl)-4-methyloxazolidin-2-one, a salt thereof, or a solvate thereof to a subject in need thereof.

39. A method for preventing and/or treating obesity, comprising:
administering a compound expressed by the formula (I), a salt thereof, or a solvate thereof: 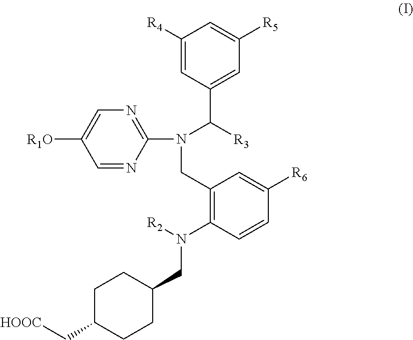
wherein
R₁ is a C₁₋₆ alkylthio C₁₋₆ alkyl group, a C₁₋₆ alkylsulfinyl C₁₋₆ alkyl group or a C₁₋₆ alkylsulfonyl C₁₋₆ alkyl group;
R₂ is a hydrogen atom or an optionally halogen atom-substituted C₁₋₆ alkyl group;
R₃ is an optionally halogen atom-substituted C₁₋₆ alkyl group;
R₄ and R₅, which are the same or different, are each independently a hydrogen atom, a halogen atom, an optionally halogen atom-substituted C₁₋₆ alkyl group, an optionally halogen atom-substituted C₁₋₆ alkoxy group, or a cyano group; and
R₆ is a hydrogen atom, a halogen atom, an optionally halogen atom-substituted C₁₋₆ alkyl group, or an optionally halogen atom-substituted C₁₋₆ alkoxy group.

40. The method of claim 39, wherein the compound is selected from the group consisting of trans-{4-[({2-[({1-[3,5-bis(trifluoromethyl)phenyl]ethyl}{5-[2-(methylsulfonyl)ethoxy]pyrimidin-2-yl}amino)methyl]-4-(trifluoromethyl)phenyl}(ethyl)amino)methyl]cyclohexyl}acetic acid, (S)-(−)-trans-{4-[({2-[({1-[3,5-bis(trifluoromethyl)phenyl]ethyl}{5-[2-(methylsulfonyl)ethoxy]pyrimidin-2-yl}amino)methyl]-4-(trifluoromethyl)phenyl}(ethyl)amino)methyl]cyclohexyl}acetic acid, (R)-(+)-trans-{4-[({2-[({1-[3,5-bis(trifluoromethyl)phenyl]ethyl}{5-[2-(methylsulfonyl)ethoxy]pyrimidin-2-yl}amino)methyl]-4-(trifluoromethyl)phenyl}(ethyl)amino)methyl]cyclohexyl}acetic acid, a salt thereof, a solvate thereof, and a combination thereof.

41. The method of claim 31, wherein the method suppresses expression of NPC1L1.

42. The method of claim 31, wherein the method suppresses expression of LIPG mRNA.

43. The method of claim 31, wherein the method suppresses expression of NPC and LIPG mRNA.

44. The method of claim 35, wherein the method suppresses NPC1L1 protein production.

45. The method of claim 35, wherein the method suppresses LIPG protein production.

46. The method of claim 35, wherein the method supresses NPC1L1 protein production and LIPG protein production.


SEQUENCE LISTING (4 sequences)

SEQ ID NO: 1 - DNA, 22 bp, Artificial sequence
  Designed DNA based on human NPC1L1 gene
caggtatggtcgcccgaagcac

SEQ ID NO: 2 - DNA, 22 bp, Artificial sequence
  Designed DNA based on human NPC1L1 gene
tgcggttgttctggaaatactg

SEQ ID NO: 3 - DNA, 20 bp, Artificial sequence
  Designed DNA based on human L1PG gene
tcaacgatgtcttgggatca

SEQ ID NO: 4 - DNA, 20 bp, Artificial sequence
  Designed DNA based on human L1PG gene
tgaagcgattggagtcagtg